(19) United States
(12) Patent Application Publication
(10) Pub. No.: US 2015/0273027 A1
(43) Pub. Date: Oct. 1, 2015

(54) LIQUID PHARMACEUTICAL COMPOSITION OF FACTOR VII POLYPEPTIDE

(71) Applicant: Novo Nordisk Healthcare AG, Zürich (CH)

(72) Inventors: Prafull S. Gandhi, Ballerup (DK); Anette Henriksen, Alleroed (DK); Charlotte C. Rossmeisl, Lynge (DK); Hanne B. Rasmussen, Hilleroed (DK); Henrik S. Andersen, Bagsvaerd (DK); Soeren E. Bjoern, Farum (DK)

(21) Appl. No.: 14/434,510

(22) PCT Filed: Oct. 10, 2013
(86) PCT No.: PCT/EP2013/071225

§ 371 Date: Apr. 9, 2015

Related U.S. Application Data
(60) Provisional application No. 61/790,957, filed on Mar. 15, 2013.
(60) Provisional application No. 61/712,187, filed on Oct. 10, 2012.

(30) Foreign Application Priority Data
Mar. 18, 2013 (EP) 13159833.6

(51) Int. Cl. A61K 38/48; A61K 47/22

(52) U.S. Cl. CPC A61K 38/4846; A61K 47/22

(57) ABSTRACT

The invention relates to a liquid, aqueous pharmaceutical composition comprising a Factor VIIa polypeptide, a buffering agent suitable for keeping pH in the range of from about 5.5 to about 8.5; and an active site stabilizing agent, which is selected from the group of: (S)-2-{2-[5-(5-carbamimidoyl-1H-benzoimidazol-2-yl)-6,2′-dihydroxy-5′-sulfamoyl-biphenyl-3-yl]acetylamino}-succinic acid or a pharmaceutically acceptable salt thereof; (R)-2-{2-[5-(5-carbamimidoyl-1H-benzoimidazol-2-yl)-6,2′-dihydroxy-5′-sulfamoyl-biphenyl-3-yl]acetylamino}-succinic acid or a pharmaceutically acceptable salt thereof; and a mixture of the (S)- and (R)-forms or pharmaceutically acceptable salts thereof. The invention further relates to said composition for treatment of a Factor VII-responsive bleeding disorder; methods for preparing the liquid composition and for stabilizing Factor VIIa in a liquid aqueous composition; an air-tight container containing the liquid, aqueous pharmaceutical composition and optionally an inert gas; and a method of treating a Factor VII-responsive bleeding disorder in a patent.

### FIELD OF THE INVENTION

The present invention relates to liquid, aqueous pharmaceutical compositions containing Factor VII(a) polypeptides; methods for preparing and using such compositions; containers containing such compositions and the use of such compositions for the treatment of a Factor VII(a)-responsive disorder. More particularly, the invention relates to liquid compositions stabilized against proteolytic, chemical and/or physical degradation.

### BACKGROUND

Blood clotting Factor VIIa (FVIIa) has proven to be an important therapeutic agent for the treatment of blood clotting disorders such as haemophilia A, haemophilia B, Glanzmann's thrombasthenia and FVII(a) deficiency.

The current commercially available, recombinant Factor VIIa formulation NovoSevenRT® (Novo Nordisk A/S, Denmark) is presented as a vial containing a freeze-dried cake of recombinant human Factor VIIa, NaCl, CaCl₂(2 H₂O), GlyGly, polysorbate 80, sucrose and mannitol. This product is reconstituted to pH 6.0 with histidine buffer immediately prior to use, thus yielding a FVIIa concentration of 1.0 mg/mL in the resulting solution.

The decision to either maintain a manufactured protein drug in a liquid, or to freeze-dry it, is usually based on the stability of the protein in those two forms. Protein stability can be affected by such factors as ionic strength, pH, temperature, repeated cycles of freezing and thawing, exposure to shear forces and the nature of the protein itself. Some of the active protein may be lost as a result of physical instability, resulting in denaturation and aggregation (both soluble and insoluble aggregate formation), as well as chemical instability, resulting in for example, hydrolysis, deamidation, and oxidation; to name just a few.

Whilst the occurrence of protein instability is widely appreciated, it is generally impossible to predict what might be the effective method to solve the particular instability-related problems of a particular protein. Instability can result in the formation of a protein by-product, or derivative, which has lowered activity, increased toxicity, and/or increased immunogenicity. Furthermore, post-translational modifications such as the gamma-carboxylation of certain glutamic acid residues in the N-terminus, or the addition of carbohydrate side chains, provide potential sites of modification during storage.

However, liquid formulations of serine proteases, such as Factor VIIa polypeptides, prompt for distinct stability concerns as they are subject to degradation by autoproteolysis by being substrates for their own catalysis (being both biological enzymes and substrates).

Formulating a protease such as a FVIIa polypeptide is a major challenge to the pharmaceutical industry because FVIIa polypeptides readily cleave other FVIIa polypeptides in the same formulation, rendering them inactive. In liquid formulations, FVIIa polypeptides can autolyse within a period of a few hours and the problem is particularly acute when the concentration of FVIIa polypeptide is high. Therefore, in creating a liquid formulation of a FVIIa polypeptide, autolysis is the greatest hurdle to be overcome.

The safety and efficacy of any protein composition is directly related to its stability. Maintaining protein stability in a liquid requires a different approach than the approach used to maintain stability in its lyophilized form because of the highly increased potential for molecular motion and therefore increased probability of molecular interactions. Maintaining stability in a concentrated solution constitutes a separate challenge because of the propensity for aggregate formation and increased protein-protein interactions at increased protein concentrations.

When developing a liquid composition, many factors are taken into consideration. Obtaining short-term (less than six months) liquid stability generally requires avoiding gross structural changes, such as denaturation and aggregation. These processes are described in the literature for a number of proteins, and many examples of stabilizing agents exist. It is well-known that an agent effective in stabilizing one protein actually acts to destabilize another. Once the protein has been stabilized against gross structural changes, developing a liquid composition for long-term stability (e.g., greater than six months) depends on further stabilizing the protein from types of degradation specific to that protein. More specific types of degradation may include, for example, disulfide bond scrambling, oxidation of certain residues, deamidation and cyclization. Although it is not always possible to pinpoint the individual degradation species, assays are developed to monitor subtle changes so as to monitor the ability of specific excipients to uniquely stabilize the protein of interest.

The pH as well as ionic strength of the liquid composition additionally needs to be in a physiologically suitable range for injection/infusion.

Factor VIIa undergoes several degradative pathways, especially autoproteolytic cleavage (clipping of the peptide backbone or “heavy chain degradation), aggregation (formation of dimeric, oligomeric and polymeric forms), and oxidation. Furthermore, precipitation and deamidation may occur. Many of these reactions can be slowed significantly by removal of water from the protein.

However, there are several advantages associated with the use of a preserved, liquid formulation rather than a freeze-dried cake that is reconstituted with a suitable liquid (e.g. WFI or a buffer) immediately prior to injection. Most notably, a preserved liquid is much more convenient to use than a freeze-dried product. The development of a liquid composition of a Factor VIIa polypeptide could eliminate reconstitution errors, thereby increasing dosing accuracy; as well as simplifying the use of the product clinically, thereby increasing patient compliance. Generally, more highly concentrated solutions allow for the administration of lower volumes, which may provide an opportunity for parenteral administration other than intravenous. Liquid compositions can thus have many advantages over freeze-dried products with regard to ease of administration and use.

Currently, no liquid-formulated FVIIa product is commercially available. It is an objective of this invention to provide a liquid Factor VIIa polypeptide pharmaceutical composition which is suitable for both storage and delivery and in which the amount of chemical and/or physical degradation products is physiologically acceptable.

WO2005016365 (Novo Nordisk Health Care AG) concerns liquid, aqueous pharmaceutical compositions comprising a Factor VIIa polypeptide, a buffering agent suitable for keeping pH in the range of 4-9, and at least one stabilizing agent comprising a —C(═NZ1R1)(—NZ2R2) motif.

EP1299354 (Aventis) describes urea and thiourea derivatives allegedly useful as inhibitors of Factor VIIa for inhibiting or reducing blood clotting or inflammatory response in the treatment of e.g. cardiovascular disease.

WO2004050637 (Pharmacyclics) describes benzoimidazole-5-carboxamidine derivatives allegedly useful as inhibitors of serine proteases including Factor VIIa for treating or preventing thromboembolic disorders, cancer or rheumatoid arthritis.

### SUMMARY

The present inventors have created liquid pharmaceutical compositions of Factor VII(a) polypeptides that exhibit improved stability. In these compositions, the Factor VIIa polypeptides are formulated with an active site stabilizing agent selected from the group of:

(S)-2-{2-[5-(5-carbamimidoyl-1H-benzoimidazol-2-yl)-6,2′-dihydroxy-5′-sulfamoyl-biphenyl-3-yl]acetylamino}-succinic acid; (R)-2-{2-[5-(5-carbamimidoyl-1H-benzoimidazol-2-yl)-6,2′-dihydroxy-5′-sulfamoyl-biphenyl-3-yl]acetylamino}-succinic acid; and mixtures of the (S)- and (R)-forms.

Thus, one aspect of the present invention relates to a liquid, aqueous pharmaceutical composition comprising a Factor VIIa polypeptide, a buffering agent suitable for keeping pH in the range of from about 5.5 to about 8.5; and an active site stabilizing agent, which is 2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof.

In another aspect, the present invention relates to a liquid pharmaceutical composition comprising a Factor VIIa polypeptide, a buffering agent suitable for keeping pH in the range of from about 5.5 to about 8.5; and an active site stabilizing agent, which is 2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof, for treatment of a Factor VII-responsive bleeding disorder.

In another aspect, the present invention relates to a method for preparing the liquid composition, comprising the step of providing the Factor VIIa polypeptide in a solution comprising a buffering agent suitable for keeping pH in the range of from about 5.5 to about 8.5 and an active site stabilizing agent, which is 2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof.

In another aspect, the present invention relates to a method for stabilizing Factor VIIa in a liquid aqueous composition, comprising the step of providing the Factor VIIa polypeptide in a solution comprising a buffering agent suitable for keeping pH in the range of from about 5.5 to about 8.5 and an active site stabilizing agent, which is 2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof.

In another aspect, the present invention relates to an air-tight container containing the liquid, aqueous pharmaceutical composition of the invention and optionally an inert gas.

In another aspect, the present invention relates to a method of treating a Factor VII-responsive bleeding disorder in a patent in need of such treatment, comprising administering to the patient a therapeutically effective amount of a liquid pharmaceutical composition as described above and a pharmaceutically acceptable carrier.

### DESCRIPTION

Factor VIIa is a serine protease having autoproteolytic properties, i.e. is subject to degradation by autolysis. Especially, the peptide bonds between amino acid residues 315-316 and 290-291 are readily cleaved during storage in solution (numbering referring to sequence of human wild-type FVIIa, SEQ ID NO 1). This cleavage is referred to as “heavy chain degradation”. Factor VIIa has its enzymatic optimum at pH 7.5 and has a low activity at pH below 5.5.

Besides autolytic cleavage, Factor VIIa undergoes several general degradative pathways, especially aggregation (formation of dimeric, oligomeric and polymeric forms), deamidation and oxidation.

Formulating FVIIa in a liquid composition is difficult particularly due to the autoproteolytic properties. However, also the additional, more general degradation pathways should be taken into consideration when storing FVIIa in solution; for example, oxidation may need to be addressed by inclusion of an anti-oxidant or reduction of the oxygen partial pressure by overlay of nitrogen or an inert gas.

One way to prevent autoproteolytic cleavage of FVIIa in liquid compositions is by non-covalent inhibition of the active site by introducing an active site stabilizing agent in the form of a FVIIa inhibitor to a solution including FVIIa. Such an active site stabilizing agent, however, must be released from the FVIIa molecule after injection, hereby releasing active FVIIa into the blood stream. Furthermore, the active site stabilizing agent should be present in a concentration with a desirable safety profile and it should preferably have no biological effect per se in the administered concentration in the dosing regimen (as characteristic for an excipient).

It is highly desirable to identify and introduce a FVIIa active site stabilizing agent that fulfils the desired liquid composition concept of:
(i) maintaining stability of the FVIIa molecule (minimising autoproteolysis and general protein degradation, and
(ii) maintaining bioactivity of the FVIIa molecule (keeping a similar bioactivity, including PK values, as FVIIa without active site stabilizing agent bound), and
(iii) ensuring a proper safety profile of the active site stabilizing agent (keeping in mind that this agent is a biologically active molecule in itself).

Thus, a major challenge lies in identifying an active site stabilizing agent which balance all three “factors”, i.e. at the same time optimize FVIIa stability, FVIIa bioactivity and safety of the active site stabilizing agent.

It is thus highly desirable to identify and introduce an active site stabilizing agent (i.e., an inhibitor of FVIIa enzymatic activity), which fulfils the following conditions:
a) At a non-toxic concentration (of the active site stabilizing agent) binds to FVIIa with a dissociation constant low enough (“strong binding”) to shift the equilibrium between the free FVIIa form and the bound FVIIa form (FVIIa+active site stabilizing agent⇄FVIIa:active site stabilizing agent) towards complete complex formation when the FVIIa composition is stored in the vial, and
b) At the same given concentration and dissociation constant releases FVIIa when injected in vivo, i.e. shifts the equilibrium towards the free forms of FVIIa and active site stabilizing agent.

In biochemistry and pharmacology, a dissociation constant (K_{d}) is a specific type of equilibrium constant that measures the propensity of a larger species to separate (dissociate) reversibly into smaller components, as when two molecules bound together by non-covalent forces falls apart into the component molecules. The dissociation constant is the inverse of the association constant (binding constant).

The dissociation constant is the dissociation constant of a protein-inhibitor complex Ki=[P][I]/[C], where [P], [I] and [C] represent the molar concentrations of the protein, inhibitor and complex, respectively. K is commonly used to describe the affinity between a ligand (L) and a protein (P) i.e. how tightly a ligand binds to a particular protein. Ligand-protein affinities are influenced by non-covalent intermolecular interactions between the two molecules such as hydrogen bonding, electrostatic interactions, hydrophobic and Van der Waals forces. They can also be affected by high concentrations of other macromolecules.

The formation of a ligand-protein complex (C) can be described by a two-state process C⇄P+L. The corresponding dissociation constant is defined K_{d}=[P][L]/[C], where [P], [L] and [C] represent the molar concentrations of the protein, ligand and complex, respectively. The dissociation constant has molar units (M). The Kd corresponds to the concentration of ligand at which half the protein molecules are bound to ligand, e.g. the concentration of ligand at which the concentration of protein with ligand bound [C], equals the concentration of protein with no ligand bound [P]. The smaller the dissociation constant, the more tightly bound the ligand is, or the higher the affinity between ligand and protein. For example, a ligand with a nanomolar (nM) dissociation constant binds more tightly to a particular protein than a ligand with a micromolar (M) dissociation constant.

Furthermore, the concentration of FVIIa administered should be at a concentration allowing administration of an effective dose for treatment of haemophilia in a desirable volume for the given route of administration, such as, e.g., a volume of 1-20 mL for i.v. injection in an adult, preferably 1-5 mL or even 2-3 mL.

The storage temperature of a ready-to-use formulation can vary between 2 and 45° C. Especially at storage temperatures above or equal to e.g. 20° C., the challenge of how to make a stable liquid formulation is increased.

The present invention resides in the development of a novel stabilized liquid aqueous pharmaceutical composition comprising a Factor VIIa polypeptide. More specifically, the liquid, aqueous pharmaceutical composition comprises an active site stabilizing agent selected from the group of:

(S)-2-{2-[5-(5-carbamimidoyl-1H-benzoimidazol-2-yl)-6,2′-dihydroxy-5′-sulfamoyl-biphenyl-3-yl]acetylamino}-succinic acid; (R)-2-{2-[5-(5-carbamimidoyl-1H-benzoimidazol-2-yl)-6,2′-dihydroxy-5′-sulfamoyl-biphenyl-3-yl]acetylamino}-succinic acid; a mixture of the (S)- and (R)-forms; and pharmaceutically acceptable salts thereof.

These active site stabilizing agents fulfil the above described requirements for a non-covalent stabilizer for liquid formulation of FVIIa even at storage temperatures equal to or above 20° C. for one month or above.

Active Site Stabilizing Agent

In one embodiment of the invention, the active site stabilizing agent is (S)-2-{2-[5-(5-carbamimidoyl-1H-benzoimidazol-2-yl)-6,2′-dihydroxy-5′-sulfamoyl-biphenyl-3-yl]acetylamino}-succinic acid (Formula I) or a pharmaceutically acceptable salt thereof.

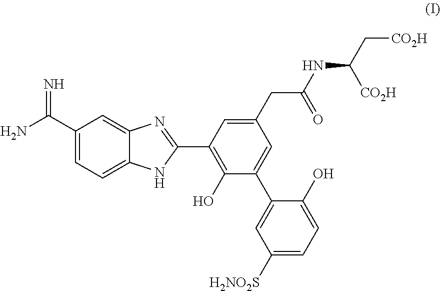 [SMILES: N=C(N)c1ccc2[nH]c(-c3cc(CC(=O)N[C@@H](CC(=O)O)C(=O)O)cc(-c4cc(S(N)(=O)=O)ccc4O)c3O)nc2c1]

In another embodiment, the active site stabilizing agent is (R)-2-{2-[5-(5-carbamimidoyl-1H-benzoimidazol-2-yl)-6,2′-dihydroxy-5′-sulfamoyl-biphenyl-3-yl]acetylamino}-succinic acid (Formula II), or a pharmaceutically acceptable salt thereof.

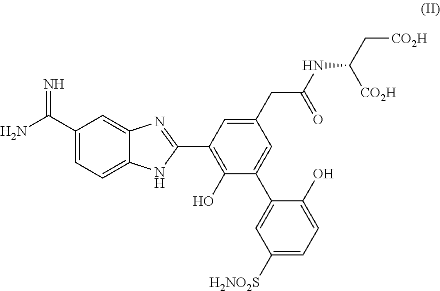 [SMILES: N=C(N)c1ccc2[nH]c(-c3cc(CC(=O)N[C@H](CC(=O)O)C(=O)O)cc(-c4cc(S(N)(=O)=O)ccc4O)c3O)nc2c1]

In yet another embodiment, the active site stabilizing agent is a mixture of (S)-2-{2-[5-(5-carbamimidoyl-1H-benzoimidazol-2-yl)-6,2′-dihydroxy-5′-sulfamoyl-biphenyl-3-yl]acetylamino}-succinic acid or a pharmaceutically acceptable salt thereof, and (R)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof.

Pharmaceutically acceptable salts include salts of acidic or basic groups present. Pharmaceutically acceptable acid addition salts include, but are not limited to, hydrochloride, hydrobromide, hydroiodide, nitrate, sulfate, bisulfate, phosphate, acid phosphate, acetate, lactate, salicylate, citrate, tartrate, pantothenate, bitartrate, ascorbate, succinate, maleate, fumarate, gluconate, glucaronate, saccharate, formate, benzoate, glutamate, methanesulfonate, ethanesulfonate, benzensulfonate, and p-toluenesulfonate salts.

Suitable base salts include, but are not limited to, calcium, magnesium, potassium, sodium, and manganese salts.

The concentration of the active site stabilizing agent(s) depends on the desired concentration of Factor VIIa in the composition ([FVIIa]). The active site stabilizing agent should preferably be present in a small excess compared to Factor VIIa. A limited excess of active site stabilizing agent is desirable to avoid unwanted side effects of the stabilizer. Thus, the active site stabilizing agent should be present in the composition in an excess of above 5 μM compared to the Factor VIIa concentration, i.e.,

[active site stabilizing agent]≧[FVIIa]+5 μM.

The concentration of the active site stabilizing agent should preferably not exceed 2.5 times the concentration of FVIIa present.

In different embodiments, the active site stabilizing agent is present in an excess of 5.5-100 μM, or 6-100 μM, or 6-75 μM, or 6-50 μM, or 6-30 μM, or 6-10 μM, or 10-100 μM, or 10-75 μM, or 10-50 μM, or 10-30 μM, or 30-50 μM, or 20-40 μM compared to the concentration of Factor VIIa, or the active site stabilizing agent is present in an excess of ≧6 μM, or ≧7 μM, or ≧10 μM, or ≧20 μM, or ≧30 μM, or ≧40 μM, or ≧50 μM compared to the concentration of Factor VIIa. In one series of embodiments, the Factor VIIa is rhFVIIa or SF-rhFVIIa, and the active site stabilizing agent is present in an excess of 5.5-50 μM, or 5.5-40 μM, or 5.5-30 μM, or 5.5-10 μM, or 6-50 μM, or 6-40 μM, or 6-30 μM, or 6-10 μM compared to the concentration of Factor VIIa.

The concentration of active site stabilizing agent(s) relative to Factor VIIa may also be given by the ratio between the concentrations (μM) of the active site stabilizing agent and FVIIa, however with the proviso that the concentration of active site stabilizing agent is more than 5 μM in excess of the concentration of FVIIa.

Thus, in various embodiments, the molar ratio between the active site stabilizing agent and FVIIa polypeptide ([active site stabilizing agent]:[FVIIa]) is: ≧1.1, or ≧1.25, or ≧1.5, or ≧1.75, or in the range of 1.1-10, or in the range of 1.25-10, or in the range of 1.5-10, or in the range of 1.75-10, or in the range of 1.1-5, or in the range of 1.25-5, or in the range of 1.5-5, or in the range of 1.25-2, or in the range of 1.75-5, or about 1.25, or about 1.5, or about 1.75, or about 2, or about 2.5. In certain embodiments, the molar ratio between the active site stabilizing agent and FVIIa polypeptide ([active site stabilizing agent]:[FVIIa]) is ≧1.5 or ≧1.75.

In one embodiment, the composition of the invention comprises FVIIa in a concentration of 40 μM and the active site stabilizing agent (S)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof, in a concentration of 60 μM.

In another embodiment, the composition of the invention comprises FVIIa in a concentration of 40 μM and the active site stabilizing agent (R)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof, in a concentration of 60 μM.

In another embodiment, the composition of the invention comprises FVIIa in a concentration of 40 μM and the active site stabilizing agent (S)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof, in a concentration of 75 μM.

In another embodiment, the composition of the invention comprises FVIIa in a concentration of 40 μM and the active site stabilizing agent (R)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof, in a concentration of 75 μM.

In other embodiments, the composition of the invention comprises FVIIa in a concentration of 40 μM and a mixture of (S)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof, and (R)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof; wherein the concentration of the mixture is 60 μM or 75 μM, respectively.

In another embodiment, the composition of the invention comprises FVIIa in a concentration of 40 μM and the active site stabilizing agent (S)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof, in a concentration of 70 μM.

In another embodiment, the composition of the invention comprises FVIIa in a concentration of 40 μM and the active site stabilizing agent (R)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof, in a concentration of 70 μM.

In other embodiments, the composition of the invention comprises FVIIa in a concentration of 40 μM and a mixture of (S)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof, and (R)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof; wherein the concentration of the mixture is 60 μM or 70 μM, respectively.

In another embodiment, the composition of the invention comprises FVIIa in a concentration of 100 μM and the active site stabilizing agent (S)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof, in a concentration of 150 μM.

In another embodiment, the composition of the invention comprises FVIIa in a concentration of 100 μM and the active site stabilizing agent (R)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof, in a concentration of 150 μM.

In another embodiment, the composition of the invention comprises FVIIa in a concentration of 100 μM and a mixture of (S)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof, and (R)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof; wherein the concentration of the mixture is 150 μM.

In another embodiment, the composition of the invention comprises FVIIa in a concentration of 200 μM and the active site stabilizing agent (S)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof, in a concentration of 210-350 μM.

In another embodiment, the composition of the invention comprises FVIIa in a concentration of 200 μM and the active site stabilizing agent (R)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof, in a concentration of 210-350 μM.

In another embodiment, the composition of the invention comprises FVIIa in a concentration of 200 μM and a mixture of (S)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof, and (R)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof; wherein the concentration of the mixture is 210-350 μM.

In addition to the components of Factor VIIa and active site stabilizing agent, the liquid, aqueous pharmaceutical composition may comprise additional components beneficial for the preparation, formulation, stability, or administration of the composition.

Divalent Metal Ion

In one embodiment, the composition of the present invention also contains a divalent metal ion selected from the group of Ca²⁺, Mg²⁺, and Mn²⁺. The metal ions may, for example, be provided in the form of a salt selected from the group of: calcium chloride, calcium acetate, calcium gluconate, calcium laevulate, manganese(II) chloride, magnesium chloride, magnesium acetate, magnesium gluconate, magnesium laevulate, and magnesium salts of strong acids.

In different embodiments, the divalent metal ion is present in a concentration of ≧2 mM, or ≧5 mM, or ≧10 mM, or in the range of 2-100 mM, or in the range of 2-50 mM, or in the range of 2-20 mM, or in the range of 5-15 mM, or in the range of 6-10 mM.

In one embodiment, the divalent metal ion is Ca²⁺. In various further embodiments, the concentration of calcium ions in the liquid composition is: ≧2 mM, or ≧5 mM, or ≧10 mM, or in the range of 2-100 mM, or in the range of 2-50 mM, or in the range of 10-50 mM, or in the range of 2-20 mM, or in the range of 5-10 mM, or in the range of 5-15 mM.

In various embodiments, the pH of the liquid composition is: in the range of 5.5-8.5, or 6.0-8.5, or 6.0-7.5, or 6.5-7.5, or 6.5-7.0, or 6.7-7.0, or 7.0-7.5.

Factor VII Polypeptides

Factor VII (FVII) is a glycoprotein primarily produced in the liver. The mature protein consists of 406 amino acid residues and is composed of four domains as defined by homology. There are an N-terminal Gla domain followed by two epidermal growth factor (EGF)-like domains and a C-terminal serine protease domain. FVII circulates in plasma as a single-chain molecule. Upon activation to activated FVII (FVIIa), the molecule is cleaved between residues Arg152 and Ile153, resulting in a two-chain protein held together by a disulfide bond. The light chain contains the Gla and EGF-like domains, whereas the heavy chain is the protease domain. FVIIa requires binding to its cell-surface cofactor tissue factor to become biologically active.

The term “Factor VII(a)” encompasses the uncleaved zymogen, Factor VII, as well as the cleaved and thus activated protease, Factor VIIa. “Factor VII(a)” includes natural allelic variants of FVII(a) that may exist and occur from one individual to another. A wild type human Factor VIIa sequence is provided in SEQ ID NO: 1, as well as in Proc. Natl. Acad. Sci. USA 1986; 83:2412-2416.

Factor VII(a) may be plasma-derived or recombinantly produced, using well known methods of production and purification. The degree and location of glycosylation, gamma-carboxylation and other post-translational modifications may vary depending on the chosen host cell and its growth conditions.

The term “Factor VII(a) polypeptide” herein refers to wild type Factor VIIa molecules as well as FVII(a) variants, FVII(a) derivatives and FVII(a) conjugates. Such variants, derivatives and conjugates may exhibit substantially the same, or improved, biological activity relative to wild-type human Factor VIIa.

The term “FVII(a) variant”, as used herein, is intended to designate Factor FVII having the sequence of SEQ ID NO: 1, wherein one or more amino acids of the parent protein have been substituted by another amino acid and/or wherein one or more amino acids of the parent protein have been deleted and/or wherein one or more amino acids have been inserted in the parent protein and/or wherein one or more amino acids have been added to the parent protein. Such addition can take place either at the N-terminal end or at the C-terminal end of the parent protein or both. The “analogue” or “analogues” within this definition still have FVII activity in its activated form. In one embodiment a variant is at least 90% identical with the sequence of SEQ ID NO: 1. In another embodiment a variant is at least 95% identical with the sequence of SEQ ID NO: 1. As used herein, any reference to a specific position refers to the corresponding position in SEQ ID NO: 1.

Non-limiting examples of FVII(a) variants that have substantially the same or increased proteolytic activity compared to recombinant wild type human Factor VII(a) include those disclosed in WO 01/83725, WO 02/22776, WO 02/077218, WO 03/027147, WO 03/037932, WO 04/029090, WO 05/024006, and EP 05108713.8, U.S. Pat. No. 7,173,000 B2; and JP4451514 B2.

The term “Factor VII(a) derivative” as used herein, is intended to designate a FVII polypeptide that exhibits substantially the same or improved biological activity relative to wild-type Factor VIIa, in which one or more of the amino acids of the parent peptide have been genetically and/or chemically and/or enzymatically modified, such as by alkylation, glycosylation, PEGylation, acylation, ester formation, disulfide bond formation, or amide formation.

The term “PEGylated human Factor VII(a)” refers to a human Factor VII(a) polypeptide, to which a PEG molecule has been conjugated. Such a PEG molecule may be attached to any part of the Factor VIIa polypeptide, including any amino acid residue or carbohydrate moiety of the Factor VIIa polypeptide. This includes but is not limited to PEGylated human Factor VIIa, cysteine-PEGylated human Factor VIIa and variants thereof. Non-limiting examples of Factor VII derivatives includes glycoPEGylated FVII(a) derivatives as disclosed in WO 03/031464 and WO 04/099231 and WO 02/077218,

The term “cysteine-PEGylated human Factor VII(a)” refers to a Factor VII(a) polypeptide in which a PEG molecule is conjugated to a sulfhydryl group of a cysteine that has been introduced into said human Factor VIIa.

The term “improved biological activity” refers to FVII(a) polypeptides that exhibit i) substantially the same or increased proteolytic activity compared to recombinant wild type human Factor VIIa in the presence and/or absence of tissue factor or ii) to FVII(a) polypeptides with substantially the same or increased TF affinity compared to recombinant wild type human Factor VIIa or iii) to FVII(a) polypeptides with substantially the same or increased half-life in plasma compared to recombinant wild type human Factor VIIa, or iv) to FVII(a) polypeptides with substantially the same or increased affinity for the activated platelet.

The biological activity of Factor VIIa in blood clotting derives from its ability to (i) bind to Tissue Factor (TF) and (ii) catalyze the proteolytic cleavage of Factor IX or Factor X to produce activated Factor IX or X (Factor IXa or Xa, respectively).

For the purposes of the invention, biological activity of Factor VII polypeptides (“Factor VII biological activity”) may be quantified by measuring the ability of a preparation to promote blood clotting, cf. Assay 1 described herein. Alternatively, Factor VIIa biological activity may be quantified by (i) measuring the ability of Factor VIIa or a Factor VII-related polypeptide to produce activated Factor X (Factor Xa) in a system comprising TF embedded in a lipid membrane and Factor X. (Persson et al., J. Biol. Chem. 272:19919-19924, 1997); or (ii) measuring the physical binding of Factor VIIa or a Factor VII-related polypeptide to TF using an instrument based on surface plasmon resonance (Persson, FEBS Letts. 413:359-363, 1997).

### SEQ ID NO 1: Wild Type Human Coagulation Factor VII

anaflyylrpgslyryckyyqcsfyyaryifkdayrtklfwisysdgdqcasspcqnggsckdqlqsyicfclpafegrnc ethkddqlicvnenggceqycsdhtgtkrscrchegyslladgvsctptveypcgkipilekrnaskpqgrivggkvcpkgecpwq vlllvngaqlcggtlintiwvvsaahcfdkiknwrnliavlgehdlsehdgdeqsrrvaqviipstyvpgttnhdiallrlhqpvvltdhv vplclpertfsertlafvrfslvsgwgqlldrgatalelmvlnvprlmtqdclqqsrkvgdspniteymfcagysdgskdsckgdsggp hathyrgtwyltgivswgqgcatvghfgvytrvsqyiewlqklmrseprpgvllrapfp

(γ designating gamma-carboxyglutamic acid (Gla))

In various embodiments, the Factor VIIa polypeptide is: human Factor VIIa (hFVIIa), recombinantly made human Factor VIIa (rhFVIIa), recombinantly made serum-free Factor VIIa (sf-rFVIIa), recombinantly made serum-free human Factor VIIa (sf-rhFVIIa) (“serum-free”: made recombinantly under serum-free culturing conditions).

In some embodiments, Factor VIIa is made by any suitable manufacturing process. In one embodiment, the Factor VII polypeptide is made by a serum-free manufacturing process according to U.S. Pat. No. 6,903,069 (incorporated by reference in its entirety).

In some embodiments, the Factor VIIa polypeptide is: a Factor VIIa sequence variant, a Factor VIIa derivative.

In different embodiments of wild-type Factor VIIa, the polypeptide is: human Factor VIIa (hFVIIa), recombinantly made human Factor VIIa (rhFVIIa), recombinantly made serum-free Factor VIIa (sf-rFVIIa), recombinantly made serum-free human Factor VIIa (sf-rhFVIIa) (“serum-free”: made recombinantly under serum-free culturing conditions).

In different embodiments, the Factor VIIa polypeptide is present in the liquid composition in a concentration of: About 0.3-200 mg/mL, or about 0.3-120 mg/mL, or about 0.5-100 mg/mL, or about 0.5-20 mg/mL, or about 1-10 mg/mL, or about 1-5.5 mg/mL, or about 2-20 mg/mL, or about 2-15 mg/mL, or about 2-10 mg/mL, or about 2-5.5 mg/mL, or about 5-15 mg/mL, or about 2 mg/mL, or about 5 mg/mL, or about 10 mg/mL.

Factor VIIa concentration is conveniently expressed as mg/mL or as IU/mL, with 1 mg usually representing 43,000-56,000 IU or more. Factor VIIa has a molecular weight of about 52 kDa. Thus, a concentration of 1 mg/mL of FVIIa corresponds to a molar concentration of about 20 μM FVIIa.

The biological effect of the pharmaceutical composition is mainly ascribed to the presence of the Factor VIIa polypeptide, although other active ingredients may be included in combination with the Factor VIIa polypeptide.

Buffering Agent

In order to render the liquid, aqueous pharmaceutical composition useful for direct parenteral administration to a mammal such as a human, it is normally required that the pH value of the composition is held within certain limits, such as from about 5.5-8.5.

To ensure a suitable pH value under the conditions given, the pharmaceutical composition also comprises a buffering agent suitable for keeping pH in the range of from about 5.5-8.5.

The term “buffering agent” include those agents or combinations of agents that maintain the solution pH in the range from about 5.5-8.5.

In one embodiment, the buffering agent is at least one component selected from the groups consisting of acids and salts of MES, PIPES, ACES, BES, TES, HEPES, TRIS, histidine (e.g. L-histidine), imidazole, glycine, glycylglycine, glycinamide, phosphoric acid (e.g. sodium or potassium phosphate), acetic acid (e.g. ammonium, sodium or calcium acetate), lactic acid, glutaric acid, citric acid (e.g. sodium or potassium citrate), tartaric acid, malic acid, maleic acid, and succinic acid. It should be understood that the buffering agent may comprise a mixture of two or more components, wherein the mixture is able to provide and maintain a pH value in the specified range.

The concentration of the buffering agent is chosen so as to maintain the preferred pH of the solution. In various embodiments, the concentration of the buffering agent is 1-100 mM; 1-50 mM; 1-25 mM; or 2-20 mM.

In different embodiments, the pH of the composition is kept from 5.5-8.5, or 6.0-8.5, or 6.0-7.5, or 6.5-7.5, or 7.0-7.5, or 6.5-7.0, or 6.7-6.9.

In different embodiments, the buffering agent comprises histidine and/or glycylglycine.

As used in the present context, pH values specified as “about” are understood to be ±0.1, e.g. about pH 8.0 includes pH 8.0±0.1.

Surfactant

The pharmaceutical composition may also include a non-ionic surfactant. Surfactants (also known as detergents) generally include those agents which protect the protein from air/solution interface induced stresses and solution/surface induced stresses (e.g. resulting in protein aggregation).

Typical types of non-ionic surfactants are polysorbates, poloxamers, polyoxyethylene alkyl ethers, polyethylene/polypropylene block co-polymers, polyethyleneglycol (PEG), polyxyethylene stearates, and polyoxyethylene castor oils.

Illustrative examples of non-ionic surfactants are Tween®, polysorbate 20, polysorbate 80, Brij-35 (polyoxyethylene dodecyl ether), poloxamer 188, poloxamer 407, PEG8000, Pluronic® polyols, polyoxy-23-lauryl ether, Myrj 49, and Cremophor A.

In one embodiment, the non-ionic surfactant is present in an amount of 0.005-2.0% by weight. In one embodiment, the non-ionic surfactant is a polysorbate or poloxamer. In another embodiment, the surfactant is polysorbate 80. In another embodiment, the surfactant is poloxamer 188.

Tonicity Modifying Agent

Also, the composition may further comprise a tonicity modifying agent. As used herein, the term “tonicity modifying agent” includes agents which contribute to the osmolality of the solution. The tonicity modifying agent includes at least one agent selected from the group consisting of neutral salts, amino acids, peptides of 2-5 amino acid residues, monosaccharides, disaccharides, oligo- and polysaccharides, and sugar alcohols. In some embodiments, the composition comprises two or more of such agents in combination.

By “neutral salt” is meant a salt that is neither an acid nor a base when dissolved in an aqueous solution. Non-limiting examples of neutral salts include sodium salts, potassium salts, calcium salts, and magnesium salts, such as, for example, sodium chloride, potassium chloride, calcium chloride, calcium acetate, calcium gluconate, calcium laevulate, magnesium chloride, magnesium acetate, magnesium gluconate and magnesium laevulate.

Non-limiting examples of saccharides that may be used as tonicity modifiers are: sucrose, mannitol, glucose (dextrose), and cyclodextrins.

In different embodiments, the tonicity modifying agent is selected from the group consisting of: sodium chloride, calcium chloride, sucrose, glucose, mannitol, cyclodextrin, and combinations of two or more of these.

In one embodiment, the tonicity modifying agent is sodium chloride, or a combination of sodium chloride and one or more additional agent(s) selected from the group of: calcium chloride, sucrose, glucose, mannitol, and cyclodextrin.

In different embodiments, the tonicity modifying agent is present in a concentration of at least 5 mM, or at least 10 mM, or at least 20 mM, or at least 50 mM, or at least 100 mM, or in the range of 10-200 mM, or 10-150 mM, or 30-150 mM, or 50-140 mM.

In one embodiment, the tonicity modifying agent is 50-140 mM sodium chloride. In another embodiment the tonicity modifying agent is sucrose and/or mannitol in a concentration of 20-40 mM.

In one embodiment, the composition is isotonic; in another, it is hypertonic.

The term “isotonic” means “isotonic with serum” (i.e., about 300±50 milliosmol/kg). The tonicity is meant to be a measure of osmolality of the solution prior to administration. The term “hypertonic” is meant to designate levels of osmolality above the physiological level of serum, such as levels above 300±50 milliosmol/kg.

Antioxidant

The active site stabilizing agents with formula I and II may themselves exhibit an antioxidative effect as the compounds are able to undergo oxidation. As a consequence, the used active site stabilizing agent may thus protect the factor VIIa molecule against oxidation. However, in a further embodiment of the invention, the composition further comprises an antioxidant. In different embodiments, the antioxidant is selected from the group consisting of: L-methionine, D-methionine, methionine analogues, methionine-containing peptides, methionine-homologues, cysteine, homocysteine, gluthatione, tyrosine, cystine, and cysstathionine. In different embodiments, the antioxidant is L-methionine, gluthathione, tyrosine, or a mixture of two or more of these.

The concentration of antioxidant is typically 0.1-5.0 mg/mL, such as 0.1-4.0 mg/mL, 0.1-3.0 mg/mL, 0.1-2.0 mg/ml, or 0.5-2.0 mg/mL.

For the product in which oxygen enters into a degradation reaction, the antioxidant effect can be achieved by displacing oxygen (air) from contact with the product. In particular embodiments, the composition does not include an antioxidant; instead the susceptibility of the Factor VII polypeptide to oxidation is controlled by exclusion of atmospheric air or by displacing oxygen (air) from contact with the product. This may e.g. be accomplished by saturating the liquid with either nitrogen or argon and sealing the final container after displacing the air above the product with the gas.

The use of an antioxidant may of course also be combined with the exclusion of atmospheric air. Furthermore, the composition may be protected from light; said protection may of course be combined with either or both of exclusion of atmospheric air and the use of an antioxidant.

Thus, the present invention also provides an air-tight container (e.g. a vial or a cartridge (such as a cartridge for a pen applicator)) containing a liquid, aqueous pharmaceutical composition as defined herein, and optionally an inert gas. The inert gas may be selected from the group consisting of nitrogen or argon. The container (e.g. vial or cartridge or syringe) is typically made of glass or plastic, in particular glass, optionally closed by a rubber septum or other closure means allowing for penetration with preservation of the integrity of the pharmaceutical composition. In a further embodiment, the container is a vial or cartridge enclosed in a sealed bag, e.g. a sealed plastic bag, such as a laminated (e.g. metal (such as aluminium) laminated plastic bag).

Solubilizing Agent

The composition of the invention may contain a solubilizing agent in order to facilitate the solution of the stabilizing agent. For example, at higher concentrations of Factor VIIa and therefrom following higher concentrations of stabilizing agent, inclusion of such an agent may prove beneficial. In particular, compositions having a pH below 6.5 may benefit from the inclusion of a solubilizing agent.

Non-limiting examples of solubilizing agents are: cyclodextrins, dimethyl sulfoxide (DMSO), 2-Hydroxypropyl-β-cyclodextrin (HPβCD).

Cyclodextrins are a group of structurally related natural products formed during bacterial digestion of cellulose. These cyclic oligosaccharides consist of (α-1,4)-linked α-D-glucopyranose units and contain a somewhat lipophilic central cavity and a hydrophilic outer surface. The natural α-, β- and γ-cyclodextrin (αCD, βCD and γCD) consist of six, seven, and eight glucopyranose units, respectively. Water-soluble cyclodextrin derivatives of commercial interest include the hydroxypropyl derivatives of βCD and γCD, the randomly methylated β-cyclodextrin (RMβCD), and sulfobutylether β-cyclodextrin sodium salt (SBEβCD).

Non-limiting examples of cyclodextrins include: α-Cyclodextrin (αCD), β-Cyclodextrin (βCD), 2-Hydroxypropyl-β-cyclodextrin (HPβCD), Sulfobutylether β-cyclodextrin sodium salt (SBEβCD), randomly methylated β-cyclodextrin (RMβCD), and 2-Hydroxypropyl-γ-cyclodextrin (HPγCD). in one embodiment the cyclodextrin is HPβCD and/or HPγCD.

In one embodiment, the solubilizing agent is present in a concentration of 5% (w/v).

Preservative

A preservative may be included in the composition to retard microbial growth and thereby allow “multiple use” packaging of the Factor VIIa polypeptides. Examples of preservatives include phenol, benzyl alcohol, orto-cresol, meta-cresol, para-cresol, methyl paraben, propyl paraben, benzalkonium chloride, and benzethonium chloride. The preservative is normally included at a concentration of 0.1-20 mg/mL depending on the pH range and type of preservative.

The compositions according to the present invention are useful as stable and preferably ready-to-use compositions of Factor VII polypeptides. The compositions are typically stable for at least six months, and preferably up to 36 months; when stored at temperatures ranging from 2° C. to 8° C. In one embodiment, the compositions are stable for 24 months when stored at temperatures ranging from 2° C. to 8° C. In another embodiment, the compositions are stable for 24 months when stored at temperatures ranging from 2° C. to 8° C. and for at least additional four weeks when stored at temperatures ranging from 25° C. to 30° C. The compositions are chemically and/or physically stable, in particular chemically stable, when stored for at least 6 months at from 2° C. to 8° C.

The term “stable” is intended to denote that (i) after storage for 6 months at 2° C. to 8° C. or storage for 2 weeks at 20° C. or above the composition retains at least 50% of its initial biological activity as measured by a one-stage clot assay essentially as described in Assay 1 of the present specification, or (ii) after storage for 6 months at 2° C. to 8° C., the increase in content of heavy chain degradation products is at the most 40% (w/w) of the initial content of Factor VIIa polypeptide.

The term “initial content” relates to the amount of Factor VIIa polypeptides added to a composition upon preparation of the composition.

The term “composition” and the term “formulation” are used interchangeably throughout the patent application.

In one embodiment, the stable composition retains at least 70%, such as, e.g., at least 80%, at least 85%, at least 90%, or at least 95%, of its initial biological activity after storage for 6 months at 2 to 8° C.

In different embodiments of the invention, the stable composition further retains at least 50% of its initial biological activity as measured by a one-stage clot assay essentially as described in Assay 1 of the present specification after storage for at least 30 days, such as 60 days or 90 days.

In various embodiments the increase in content of heavy chain degradation products in the stable compositions is not more than about 10%, not more than about 8%, not more than about 5%, or not more than about 3% of the initial content of Factor VIIa polypeptide. Content of heavy chain degradation products is measured as described in Assay 2, below.

The term “physical stability” of Factor VII polypeptides relates to the formation of insoluble and/or soluble aggregates in the form of dimeric, oligomeric and polymeric forms of Factor VII polypeptides as well as any structural deformation and denaturation of the molecule. Physically stable composition encompasses compositions which remains visually clear. Physical stability of the compositions is often evaluated by means of visual inspection and turbidity after storage of the composition at different temperatures for various time periods. Visual inspection of the compositions is performed in a sharp focused light with a dark background. A composition is classified as physically unstable, when it shows visual turbidity.

The term “chemical stability” is intended to relate to the formation of any chemical change in the Factor VII polypeptides upon storage in solution at accelerated conditions. Examples are hydrolysis, deamidation and oxidation as well as enzymatic degradation resulting in formation of fragments of Factor VII polypeptides. In particular, the sulphur-containing amino acids are prone to oxidation with the formation of the corresponding sulphoxides.

The term “chemically stable” is intended to designate a composition which retains at least 50% of its initial biological activity after storage for 6 months at 2 to 8° C., as measured by a one-stage clot assay (Assay 1).

In various embodiments the increase in content of oxidation/degradation products in the stable compositions is not more than about 10% (w/w), not more than about 8% (w/w), not more than about 5% (w/w), or not more than about 3% of the initial content of Factor VIIa polypeptide. Content of oxidation/degradation products is measured as described in Assay 2, below.

### VARIOUS EMBODIMENTS

In one embodiment, the FVIIa composition comprises 2-5 mg/ml FVIIa, 10-100 μM excess of stabilizing agent relative to FVIIa, 5-20 mM Ca²⁺, methionine 0.1-2.0 mg/mL, at pH 6.5-7.0. In one embodiment, the composition is protected during storage from atmospheric oxygen and/or is protected against light. The protection against oxygen may, e.g. be done by sealing the vial with an oxygen-tight seal, or filling the vial with nitrogen or an inert gas before sealing, or both. In further embodiments, the composition further comprises polysorbate or poloxamer.

In a series of embodiments, the liquid composition of the present invention comprises:

1-10 mg/mL Factor VIIa, (S)-2-{2-[5-(5-carbamimidoyl-1H-benzoimidazol-2-yl)-6,2′-dihydroxy-5′-sulfamoyl-biphenyl-3-yl]acetylamino}-succinic acid (Formula I) or a pharmaceutically acceptable salt thereof in a ratio of 1.1 μM-2.5 μM per 1 μM of Factor VIIa present; 6-50 mM Ca²⁺, 0.1-2.0 mg/mL of methionine, pH 6.5-7.5;

1-10 mg/mL Factor VIIa, (S)-2-{2-[5-(5-carbamimidoyl-1H-benzoimidazol-2-yl)-6,2′-dihydroxy-5′-sulfamoyl-biphenyl-3-yl]acetylamino}-succinic acid (Formula I) or a pharmaceutically acceptable salt thereof in a ratio of 1.1 μM-2.5 μM per 1 μM of Factor VIIa present; 6-50 mM Ca²⁺, 0.25-5 mg/mL of methionine, pH 6.5-7.5;

1-10 mg/mL Factor VIIa, (S)-2-{2-[5-(5-carbamimidoyl-1H-benzoimidazol-2-yl)-6,2′-dihydroxy-5′-sulfamoyl-biphenyl-3-yl]acetylamino}-succinic acid (Formula I) or a pharmaceutically acceptable salt thereof in a ratio of 1.1 μM-2.5 μM per 1 μM of Factor VIIa present; 6-50 mM Ca²⁺, 0.5-1.50 mg/mL of methionine, pH 6.5-7.5;

2-5 mg/mL Factor VIIa, (S)-2-{2-[5-(5-carbamimidoyl-1H-benzoimidazol-2-yl)-6,2′-dihydroxy-5′-sulfamoyl-biphenyl-3-yl]acetylamino}-succinic acid (Formula I) or a pharmaceutically acceptable salt thereof in a ratio of 1.1 μM-2.5 μM per 1 μM of Factor VIIa present; 6-50 mM Ca²⁺, 0.1-2.0 mg/mL of methionine, pH 6.5-7.5;

2-5 mg/mL Factor VIIa, (S)-2-{2-[5-(5-carbamimidoyl-1H-benzoimidazol-2-yl)-6,2′-dihydroxy-5′-sulfamoyl-biphenyl-3-yl]acetylamino}-succinic acid (Formula I) or a pharmaceutically acceptable salt thereof in a ratio of 1.1 μM-2.5 μM per 1 μM of Factor VIIa present; 6-50 mM Ca²⁺, 0.25-5 mg/mL of methionine, pH 6.5-7.5;

2-5 mg/mL Factor VIIa, (S)-2-{2-[5-(5-carbamimidoyl-1H-benzoimidazol-2-yl)-6,2′-dihydroxy-5′-sulfamoyl-biphenyl-3-yl]acetylamino}-succinic acid (Formula I) or a pharmaceutically acceptable salt thereof in a ratio of 1.1 μM-2.5 μM per 1 μM of Factor VIIa present; 6-50 mM Ca²⁺, 0.5-1.50 mg/mL of methionine, pH 6.5-7.5;

1-10 mg/mL Factor VIIa, (S)-2-{2-[5-(5-carbamimidoyl-1H-benzoimidazol-2-yl)-6,2′-dihydroxy-5′-sulfamoyl-biphenyl-3-yl]acetylamino}-succinic acid (Formula I) or a pharmaceutically acceptable salt thereof in a ratio of 1.75 μM per 1 μM of Factor VIIa present; 6-50 mM Ca²⁺, 0.1-2.0 mg/mL of methionine, pH 6.5-7.5;

1-10 mg/mL Factor VIIa, (S)-2-{2-[5-(5-carbamimidoyl-1H-benzoimidazol-2-yl)-6,2′-dihydroxy-5′-sulfamoyl-biphenyl-3-yl]acetylamino}-succinic acid (Formula I) or a pharmaceutically acceptable salt thereof in a ratio of 1.75 μM per 1 μM of Factor VIIa present; 6-50 mM Ca²⁺, 0.25-5 mg/mL of methionine, pH 6.5-7.5;

1-10 mg/mL Factor VIIa, (S)-2-{2-[5-(5-carbamimidoyl-1H-benzoimidazol-2-yl)-6,2′-dihydroxy-5′-sulfamoyl-biphenyl-3-yl]acetylamino}-succinic acid (Formula I) or a pharmaceutically acceptable salt thereof in a ratio of 1.75 μM per 1 μM of Factor VIIa present; 6-50 mM Ca²⁺, 0.5-1.50 mg/mL of methionine, pH 6.5-7.5;

2-5 mg/mL Factor VIIa, (S)-2-{2-[5-(5-carbamimidoyl-1H-benzoimidazol-2-yl)-6,2′-dihydroxy-5′-sulfamoyl-biphenyl-3-yl]acetylamino}-succinic acid (Formula I) or a pharmaceutically acceptable salt thereof in a ratio of 1.75 μM per 1 μM of Factor VIIa present; 6-50 mM Ca²⁺, 0.1-2.0 mg/mL of methionine, pH 6.5-7.5;

2-5 mg/mL Factor VIIa, (S)-2-{2-[5-(5-carbamimidoyl-1H-benzoimidazol-2-yl)-6,2′-dihydroxy-5′-sulfamoyl-biphenyl-3-yl]acetylamino}-succinic acid (Formula I) or a pharmaceutically acceptable salt thereof in a ratio of 1.75 μM per 1 μM of Factor VIIa present; 6-50 mM Ca²⁺, 0.25-5 mg/mL of methionine, pH 6.5-7.5;

2-5 mg/mL Factor VIIa, (S)-2-{2-[5-(5-carbamimidoyl-1H-benzoimidazol-2-yl)-6,2′-dihydroxy-5′-sulfamoyl-biphenyl-3-yl]acetylamino}-succinic acid (Formula I) or a pharmaceutically acceptable salt thereof in a ratio of 1.75 μM per 1 μM of Factor VIIa present; 6-50 mM Ca²⁺, 0.5-1.50 mg/mL of methionine, pH 6.5-7.5;

1.0-5.0 mg/mL Factor VIIa, 30 μM-160 μM active site stabilizing agent with formula I, 1.47 mg/mL CaCl2, 2H2O, 7.50 mg/mL NaCl, 0.5 mg/mL Methionine, 0.07 mg/mL Polysorbate, 1.55 mg/mL Histidine, 1.32 mg/mL Glycylglycine, pH 6.5-7.5;

1.0-5.0 mg/mL Factor VIIa, 30 μM-160 μM active site stabilizing agent with formula II, 1.47 mg/mL CaCl2, 2H2O, 7.50 mg/mL NaCl, 0.5 mg/mL Methionine, 0.07 mg/mL Polysorbate, 1.55 mg/mL Histidine, 1.32 mg/mL Glycylglycine, pH 6.5-7.5;

2.0 mg/mL Factor VIIa, 70 μM (0.04179 mg/mL; MW=596.57) active site stabilizing agent with formula I, (MW=596.57 g/mol), 1.47 mg/mL CaCl2, 2H2O, 7.50 mg/mL NaCl, 0.5 mg/mL Methionine, 0.07 mg/mL Polysorbate, 1.55 mg/mL Histidine, 1.32 mg/mL Glycylglycine, pH 6.5-7.5;

2.0 mg/mL Factor VIIa, 70 μM (0.04179 mg/mL; MW=596.57) active site stabilizing agent with formula I, 1.47 mg/mL CaCl2, 2H2O, 7.50 mg/mL NaCl, 0.5 mg/mL Methionine, 0.07 mg/mL Polysorbate, 1.55 mg/mL Histidine, 1.32 mg/mL Glycylglycine, pH 6.8;

In particular embodiments of the above, the listed exemplary composition further contain polysorbate or poloxamer and, optionally, cyclodextrin. In further particular embodiments hereof, Factor VIIa is human recombinant FVIIa (rhFVIIa) or serum-free human recombinant FVIIa (sf-rhFVIIa).

In further particular embodiments, the listed exemplary composition are protected during storage from atmospheric oxygen and/or are protected against light. The protection against oxygen may, e.g. be done by sealing the vial with an oxygen-tight seal, or filling the vial with nitrogen or an inert gas before sealing, or both.

Method for Preparing the Composition

In a further aspect, the invention also provides a method for preparing a liquid, aqueous pharmaceutical composition of a Factor VII polypeptide, comprising the step of providing the Factor VIIa polypeptide in a solution comprising a buffering agent suitable for keeping pH in the range of from about 5.5 to about 8.5 and an active site stabilizing agent, which is 2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof.

Methods of Use

As will be understood, the liquid, aqueous pharmaceutical compositions defined herein can be used in the field of medicine. Thus, the present invention in particular provides the liquid, aqueous pharmaceutical compositions defined herein for use as a medicament, more particular for use as a medicament for treating a Factor VII-responsive disorder.

Consequently, the present invention also provides the use of the liquid, aqueous pharmaceutical composition as defined herein for the preparation of a medicament for treating a Factor VII-responsive disorder, as well as a method for treating a Factor VII-responsive disorder, the method comprising administering to a subject in need thereof an effective amount of the liquid, aqueous pharmaceutical composition as defined herein.

The preparations of the present invention may be used to treat any Factor VII-responsive disorder, such as, e.g., bleeding disorders, including those caused by clotting Factor deficiencies (e.g., haemophilia A, haemophilia B, coagulation Factor XI deficiency, coagulation Factor VII deficiency); by thrombocytopenia or von Willebrand's disease, or by clotting Factor inhibitors (e.g. inhibitors to coagulation Factors VIII or IX), and intra-cerebral haemorrhage, or excessive bleeding from any cause. The preparations may also be administered to patients in association with surgery or other trauma or to patients receiving anticoagulant therapy. The preparations of the present invention may be used for treatment of bleedings connected with, or caused by clotting Factor deficiencies (e.g., haemophilia A, haemophilia B, coagulation Factor XI deficiency, coagulation Factor VII deficiency); by thrombocytopenia, von Willebrand's disease, Glanzmann's thrombasthenia, or by clotting Factor inhibitors (e.g. antibodies to coagulation Factors VIII or IX),

The term “effective amount” is the effective dose to be determined by a qualified practitioner, who may adjust dosages to achieve the desired patient response. Factors for consideration of dose will include potency, bioavailability, desired pharmacokinetic/pharmacodynamic profiles, condition of treatment, patient-related factors (e.g. weight, health, age, etc.), presence of co-administered medications (e.g., anticoagulants), time of administration or other factors known to a medical practitioner.

The term “treatment” is defined as the management and care of a subject, e.g. a mammal, in particular a human, for the purpose of preventing, alleviating or curing a disease or the symptoms of a disease, condition or disorder. This includes the administration of a Factor VII polypeptide to prevent the onset of the symptoms or complications, or alleviating said symptoms or complications, or eliminating the disease, condition, or disorder. Pharmaceutical compositions according to the present invention containing a Factor VII polypeptide may be administered parenterally to subjects in need of such a treatment. Non-exclusive examples of such parenteral administration are subcutaneous, intramuscular, intradermal, or intravenous injection, optionally by means of a pen-like device, a syringe, e.g. in the form of a pre-filled syringe, or an infusion pump.

### LIST OF EMBODIMENTS

1. A liquid pharmaceutical composition comprising:

A Factor VIIa polypeptide;

A buffering agent suitable for keeping pH in the range of from about 5.5 to about 8.5; and

An active site stabilizing agent, which is 2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof.

2. A composition according to embodiment 1, wherein the active site stabilizing agent is (S)-2-{2-[5-(5-carbamimidoyl-1H-benzoimidazol-2-yl)-6,2′-dihydroxy-5′-sulfamoyl-biphenyl-3-yl]acetylamino}-succinic acid or a pharmaceutically acceptable salt thereof.

3. A composition according to embodiment 1, wherein the active site stabilizing agent is (R)-2-{2-[5-(5-carbamimidoyl-1H-benzoimidazol-2-yl)-6,2′-dihydroxy-5′-sulfamoyl-biphenyl-3-yl]acetylamino}-succinic acid or a pharmaceutically acceptable salt thereof.

4. A composition according to embodiment 1, wherein the active site stabilizing agent is a mixture of (S)-2-{2-[5-(5-carbamimidoyl-1H-benzoimidazol-2-yl)-6,2′-dihydroxy-5′-sulfamoyl-biphenyl-3-yl]acetylamino}-succinic acid or a pharmaceutically acceptable salt thereof; and (R)-2-{2-[5-(5-carbamimidoyl-1H-benzoimidazol-2-yl)-6,2′-dihydroxy-5′-sulfamoyl-biphenyl-3-yl]acetylamino}-succinic acid or a pharmaceutically acceptable salt thereof.

5. A composition according to any one of embodiment 1-4, wherein the concentration of the active site stabilizing agent is >5 μM in excess of the concentration of the FVIIa polypeptide (μM)

6. A composition according to any one of embodiments 1-5, wherein the concentration of the active site stabilizing agent is from >5 μM in excess of the concentration of the FVIIa polypeptide (μM) to 2.5 times the concentration of the FVIIa polypeptide present (μM).

7. A composition according to any one of embodiments 1-6, wherein the active site stabilizing agent is present in an excess of 5.5-100 μM, or 5.5-50 μM, or 5.5-30 μM, or 5.5-10 μM, or 6-50 μM, or 6-30 μM, or 6-10 μM compared to the concentration of Factor VIIa; or the active site stabilizing agent is present in an excess of ≧20 μM, or ≧30 μM, or ≧40 μM, or ≧50 μM compared to the concentration of Factor VIIa.

8. A composition according to any one of embodiments 1-4, wherein the molar ratio between the active site stabilizing agent and FVIIa polypeptide ([active site stabilizing agent]:[FVIIa]) is: ≧1.1, or ≧1.25, or ≧1.5, or in the range of 1.1-10, or in the range of 1.25-10, or in the range of 1.5-10, or in the range of 1.1-5, or in the range of 1.25-5, or in the range of 1.5-5, or about 1.25, or about 1.5, or about 2, or about 2.5.

9. A composition according to any one of embodiments 1-5, wherein the molar ratio between the active site stabilizing agent and FVIIa polypeptide ([active site stabilizing agent]:[FVIIa]) is ≧1.25, or 1.5.

10. A composition according to any one of embodiments 1-9, wherein the Factor VII polypeptide is present in a concentration of: About 0.3-200 mg/mL, or about 0.3-120 mg/mL, or about 0.5-100 mg/mL, or about 0.5-20 mg/mL, or about 1-10 mg/mL, or about 1-5.5 mg/mL, or about 2-20 mg/mL, or about 2-15 mg/mL, or about 2-10 mg/mL, or about 2-5.5 mg/mL, or about 2 mg/mL, or about 5 mg/mL.

11. A composition according to any one of embodiments 1-10, having a pH value from 6.0-8.5, or 6.0-7.5, or 6.5-7.5, or 7.0-7.5, or 6.5-7.0.

12. A composition according to any one of embodiments 1-11, wherein the buffering agent comprises at least one component selected from the group consisting of acids and salts of MES, PIPES, ACES, BES, TES, HEPES, TRIS, histidine, imidazole, glycine, glycylglycine, glycinamide, phosphoric acid, acetic acid, lactic acid, glutaric acid, citric acid, tartaric acid, malic acid, maleic acid, and succinic acid.

13. A composition according to any one of embodiments 1-12, wherein the formulation comprises a divalent metal cation selected from the group of: Ca²⁺, Mg²⁺ and/or Mn²⁺.

14. A composition according to embodiment 13, wherein the divalent metal cation is Ca²⁺.

15. A composition according to any one of embodiments 1-14, wherein the formulation comprises an antioxidant.

16. A composition according to embodiment 15, wherein the antioxidant is methionine.

17. A composition according to any one of embodiment 1-16, wherein the formulation comprises a tonicity modifying agent.

18. A composition according to embodiment 17, wherein the tonicity modifying agent is selected from the group of: NaCl, mannitol, sucrose, or a mixture of two or more of these.

19. A composition according to any one of embodiments 1-18, wherein the formulation comprises a surfactant.

20. A composition according to embodiment 19, wherein the surfactant is selected from: polysorbate or poloxamer.

21. A composition according to any one of embodiments 1-20, wherein the formulation comprises a solubilizing agent.

22. A composition according to embodiment 21, wherein the solubilizing agent is a cyclodextrin.

23. A composition according to any one of embodiments 1-22, wherein the Factor VII polypeptide is human Factor VIIa, or recombinant human Factor VIIa or serum-free recombinant human FVIIa

24. A composition according to any one of embodiments 1-23, wherein the Factor VII polypeptide is a Factor VII sequence variant, or a Factor VII derivative.

25. A method of treating a Factor VII-responsive bleeding disorder in a patent in need of such treatment, comprising administering to the patient a therapeutically effective amount of a liquid pharmaceutical composition according to any one of embodiments 1-26 and a pharmaceutically acceptable carrier.

26. A liquid pharmaceutical composition according to embodiments 1-24 for treatment of a Factor VII-responsive bleeding disorder.

27. A liquid pharmaceutical composition according to embodiment 26, wherein said bleeding disorder is selected from the list of: haemophilia A, haemophilia B, coagulation Factor XI deficiency, coagulation Factor VII deficiency, thrombocytopenia, and Von Willebrand's disease.

28. A method for preparing a liquid pharmaceutical composition according to embodiments 1-24, comprising the step of.

Providing the Factor VIIa polypeptide in a solution comprising a buffering agent suitable for keeping pH in the range of from about 5.5 to about 8.5 and an active site stabilizing agent, which is 2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof.

29. A method for stabilizing Factor VIIa in a liquid aqueous composition, comprising the step of:

Providing the Factor VIIa polypeptide in a solution comprising a buffering agent suitable for keeping pH in the range of from about 5.5 to about 8.5 and an active site stabilizing agent, which is 2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof.

30. An air-tight container containing a liquid, aqueous pharmaceutical composition as defined in embodiments 1-24 and optionally an inert gas.

31. An air-tight container according to embodiment 30, containing an inert gas selected from the group consisting of nitrogen and argon.

### EXAMPLES

### Materials and Methods

### Abbreviations

FVII=blood coagulation Factor VII

FVIIa=blood coagulation Factor VII in its activated, two-chain (cleaved) form

rFVIIa=recombinant activated factor VII

rhFVIIa=recombinant human Factor VII in the activated form

PEG=polyethylene glycol

sf-rFVIIa (SF-rFVIIa)=serum-free recombinant Factor VII in the activated form

sf-rhFVIIa (SF-rhFVIIa)=serum-free recombinant human Factor VII in the activated form

wt-FVII=wild-type Factor VII

HPLC=high-performance liquid chromatography

RP=reverse phase

SE=size exclusion

### Preparation and Purification of Factor VII Polypeptides

Human purified Factor VIIa suitable for use in the present invention is preferably made by DNA recombinant technology, e.g. as described by Hagen et al., Proc. Natl. Acad. Sci. USA 83: 2412-2416, 1986, or as described in European Patent No. 0 200 421 (ZymoGenetics, Inc.). In some embodiments, Factor VIIa is made by any suitable manufacturing process. In one embodiment, the Factor VII polypeptide is made by serum-free manufacturing process according to U.S. Pat. No. 6,903,069 (incorporated by reference in its entirety).

Factor VII may also be produced by the methods described by Broze and Majerus, J. Biol. Chem. 255 (4): 1242-1247, 1980 and Hedner and Kisiel, J. Clin. Invest. 71: 1836-1841, 1983. These methods yield Factor VII without detectable amounts of other blood coagulation Factors. An even further purified Factor VII preparation may be obtained by including an additional gel filtration as the final purification step. Factor VII is then converted into activated Factor VIIa by known means, e.g. by several different plasma proteins, such as Factor XIIa, IX a or Xa. Alternatively, as described by Bjoern et al. (Research Disclosure, 269 September 1986, pp. 564-565), Factor VII may be activated by passing it through an ion-exchange chromatography column, such as Mono Q® (Pharmacia fine Chemicals) or the like, or by autoactivation in solution.

Factor VII variants may be produced by modification of wild-type Factor VII or by recombinant technology. Factor VII variants with altered amino acid sequence when compared to wild-type Factor VII may be produced by modifying the nucleic acid sequence encoding wild-type Factor VII either by altering the amino acid codons or by removal of some of the amino acid codons in the nucleic acid encoding the natural Factor VII by known means, e.g. by site-specific mutagenesis.

It will be apparent to those skilled in the art that substitutions can be made outside the regions critical to the function of the Factor VIIa molecule and still result in an active polypeptide. Amino acid residues essential to the activity of the Factor VII polypeptide, and therefore preferably not subject to substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for coagulant, respectively cross-linking activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labelling (see, e.g., de Vos et al., 1992, Science 255: 306-312; Smith et al., 1992, Journal of Molecular Biology 224: 899-904; Wlodaver et al., 1992, FEBS Letters 309: 59-64).

The introduction of a mutation into the nucleic acid sequence to exchange one nucleotide for another nucleotide may be accomplished by site-directed mutagenesis using any of the methods known in the art. Particularly useful is the procedure that utilizes a super-coiled, double-stranded DNA vector with an insert of interest and two synthetic primers containing the desired mutation. The oligonucleotide primers, each complementary to opposite strands of the vector, extend during temperature cycling by means of Pfu DNA polymerase. On incorporation of the primers, a mutated plasmid containing staggered nicks is generated. Following temperature cycling, the product is treated with DpnI which is specific for methylated and hemi-methylated DNA to digest the parental DNA template and to select for mutation-containing synthesized DNA. Other procedures known in the art for creating, identifying and isolating variants may also be used, such as, for example, gene shuffling or phage display techniques.

Separation of polypeptides from their cell of origin may be achieved by any method known in the art, including, without limitation, removal of cell culture medium containing the desired product from an adherent cell culture; centrifugation or filtration to remove non-adherent cells and the like.

Optionally, Factor VII polypeptides may be further purified. Purification may be achieved using any method known in the art, including, without limitation, affinity chromatography, such as, e.g., on an anti-Factor VII antibody column (see, e.g., Wakabayashi et al., J. Biol. Chem. 261:11097, 1986; and Thim et al., Biochem. 27:7785, 1988); hydrophobic interaction chromatography; ion-exchange chromatography; size exclusion chromatography; electrophoretic procedures (e.g., preparative isoelectric focusing (IEF), differential solubility (e.g., ammonium sulfate precipitation), or extraction and the like. See, generally, Scopes, Protein Purification, Springer-Verlag, New York, 1982; and Protein Purification, J. C. Janson and Lars Ryden, editors, VCH Publishers, New York, 1989. Following purification, the preparation preferably contains less than 10% by weight, more preferably less than 5% and most preferably less than 1%, of non-Factor VII polypeptides derived from the host cell.

Factor VII polypeptides may be activated by proteolytic cleavage, using Factor XIIa or other proteases having trypsin-like specificity, such as, e.g., Factor IXa, kallikrein, Factor Xa, and thrombin. See, e.g., Osterud et al., Biochem. 11:2853 (1972); Thomas, U.S. Pat. No. 4,456,591; and Hedner et al., J. Clin. Invest. 71:1836 (1983). Alternatively, Factor VII polypeptides may be activated by passing it through an ion-exchange chromatography column, such as Mono Q® (Pharmacia) or the like, or by autoactivation in solution. The resulting activated Factor VII polypeptide may then be formulated and administered as described in the present application.

Factor VII derivatives such as glycoPEGylated FVIIa may e.g. be made by remodelling and glycoconjugation of peptides, for example as disclosed in WO 03/031464 and WO 04/099231 and WO 02/077218.

Assays Suitable for Determining the Biological Activity of Factor VII Polypeptides

Factor VII polypeptides useful in accordance with the present invention may be selected by suitable assays that can be performed as simple preliminary in vitro tests.

### One-Stage Coagulation Assay (Clot Assay) (Assay 1)

The clot assay is used to assess the ability of Factor VIIa polypeptides to make blood clot. For this purpose, the sample to be tested is diluted in 50 mM PIPES-buffer, pH 7.2, 1% BSA or other relevant buffer with similar properties and 40 μl is incubated with 40 μl of Factor VII deficient or depleted plasma and 80 μl of human recombinant tissue factor containing 10 mM Ca2+ and synthetic phospholipids. Coagulation times (clotting times) are measured and compared to a standard curve using a reference standard in a parallel line assay.

Assays Suitable for Measuring Degradation of Factor VII Polypeptides

Measurement of rFVIIa Fragmentation and Oxidation Products (Assay 2)

Heavy chain fragmentation and oxidation products of rFVIIa were determined by reverse phase HPLC. The RP-HPLC was run on a proprietary 4.5×250 mm butyl-bonded silica column with a particle size of 5 μm and pore size 300 Å. Column temperature: 70° C. A-buffer: 0.1% v/v trifluoracetic acid. B-buffer: 0.09% v/v trifluoracetic acid, 80% v/v acetonitrile. The column was eluted with a gradient elution from X to (X+13)% B in 30 minutes. X was adjusted so that FVIIa elutes with a retention time of approximately 26 minutes. Flow rate: 1.0 mL/min. Detection: 214 nm. Load: 20-25 μg FVIIa.

Measurement of rFVIIa Aggregation Products (Assay 3)

To determine the content of aggregated rFVIIa species (dimers, oligomers), the rFVIIa samples were subjected to analytical SE-HPLC. The analytical SE-HPLC was performed using a Waters Protein Pack 300 SW (80013) (7.5 mm×300 mm) column. Column temperature: 23°-25° C. The mobile phase was 0.2 M ammonium sulphate, 5% (v/v) 2-propanol buffer with a flow rate of 0.5 mL/min. Column load: 10 μg-25 μg SF-FVIIa. UV-detection was at 215 nm.

Measurement of rFVIIa Deamidation Products (Assay 4)

The content of deamidated rFVIIa products in the working examples below was described by peptide mapping. The absolute values reported may only be used as indicative and approximate estimates as the method has not been developed to accurately quantify this impurity.

Trypsin digestion was performed on the native protein, and the resulting peptides were analysed by RP-HPLC after digestion. Initially, samples were desalted into digestion buffer containing 2 M Urea, 50 mM Tris, 2 mM CaCl₂ and 8 mM methylamine, pH 7.8 using a NAPS column (GE Healthcare). The buffer-exchanged rFVIIa was diluted to 0.15 mg/mL using digestion buffer. Trypsin solubilised in resuspension buffer (Promega) was used for rFVIIa digestion with a trypsin to rFVIIa ratio of 1:10 (w/w). The samples were incubated at 40° C. for 6 hours. After incubation, the sample were added trifluoracetic acid to a final concentration of 2% (v/v). Samples were frozen immediately to stop the enzymatic reaction or analysed directly by RP-HPLC.

For RP-HPLC, the peptides generated by trypsin digestion were separated using a Jupiter C18 (3 μm, 2×150 mm, Phenomenex) column. The column temperature was 45° C., flow rate 0.25 mL/min, peptides were detected at 215 nm. A volume of 18 μL sample was injected. Solvents were: A-buffer: 0.06% trifluoracetic acid in water and B-buffer: 0.055% trifluoracetic acid in 90% acetonitrile. Separation was performed using linear gradients of 2.0-29.0% B-buffer over 82 min, 29.0-43.0 B-buffer over 14 min, 43.0-78.0 B-buffer over 35 min followed by 5 min using 100% B-buffer.

Synthesis of Stabilizing Agents

Methods, including suitable starting materials, for making the compounds acting as stabilizing agents according to the present invention are described in U.S. Pat. No. 7,479,502 B2 (published as WO 2004/050637 on 17 Jun., 2004); see in particular Example 17 (column 109-113) specifically referring to the compound in column 111, lines 8-15. Furthermore, WO 2005/118554 (published on 15 Dec., 2005) describes methods for making the compounds, see Examples, page 36-54, in particular Examples 1 and 2 (page 48-54)

The compound (S)-2-{2-[5-(5-carbamimidoyl-1H-benzoimidazol-2-yl)-6,2′-dihydroxy-5′-sulfamoyl-biphenyl-3-yl]acetylamino}-succinic acid has the formula I:

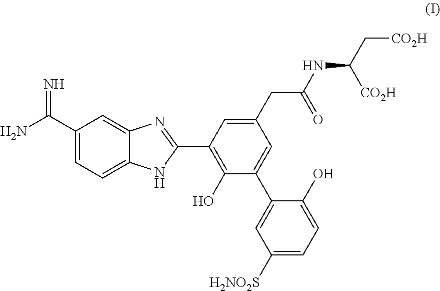 [SMILES: N=C(N)c1ccc2[nH]c(-c3cc(CC(=O)N[C@@H](CC(=O)O)C(=O)O)cc(-c4cc(S(N)(=O)=O)ccc4O)c3O)nc2c1]

The compound (R)-2-{2-[5-(5-carbamimidoyl-1H-benzoimidazol-2-yl)-6,2′-dihydroxy-5′-sulfamoyl-biphenyl-3-yl]acetylamino}-succinic acid has the formula II:

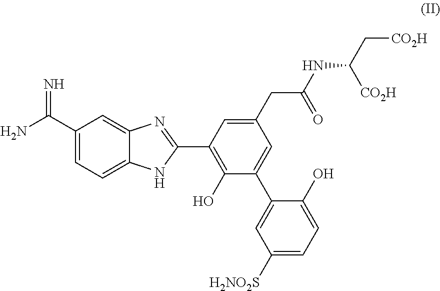 [SMILES: N=C(N)c1ccc2[nH]c(-c3cc(CC(=O)N[C@H](CC(=O)O)C(=O)O)cc(-c4cc(S(N)(=O)=O)ccc4O)c3O)nc2c1]

### WORKING EXAMPLES

### Example 1

### Synthesis of (S)-2-{2-[5-(5-carbamimidoyl-1H-benzimidazol-2-yl)-6,2′-dihydroxy-5′-sulfamoylbiphenyl-3-yl]acetylamino}succinic acid (18)

Total synthesis of (S)-2-{2-[5-(5-carbamimidoyl-1H-benzimidazol-2-yl)-6,2′-dihydroxy-5′-sulfamoylbiphenyl-3-yl]acetylamino}succinic acid (18) was done as described in US2008/0275250 A1 page 16 through 23 and as depicted in scheme 1.

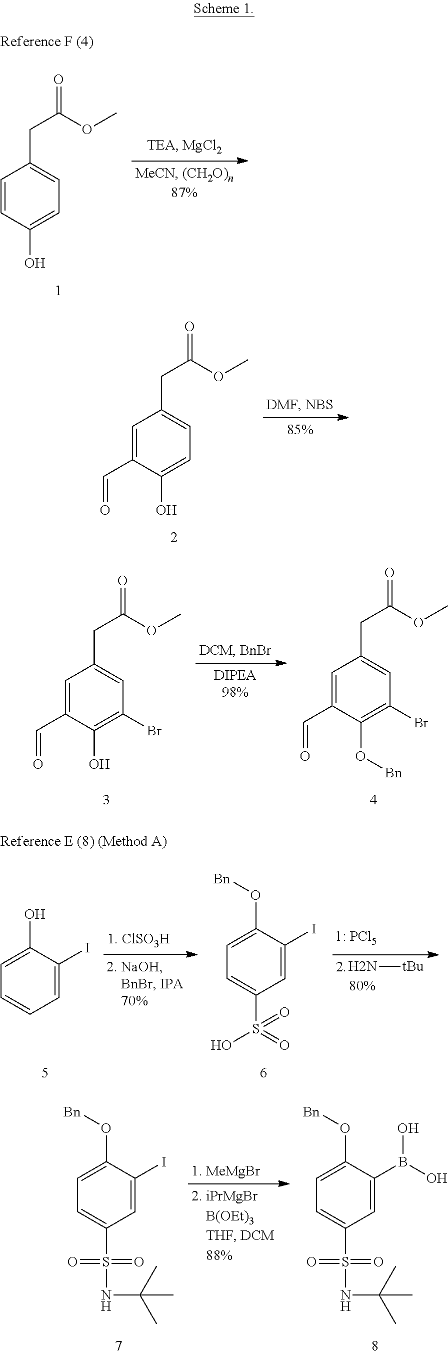

Example 1 from US2008/0275250

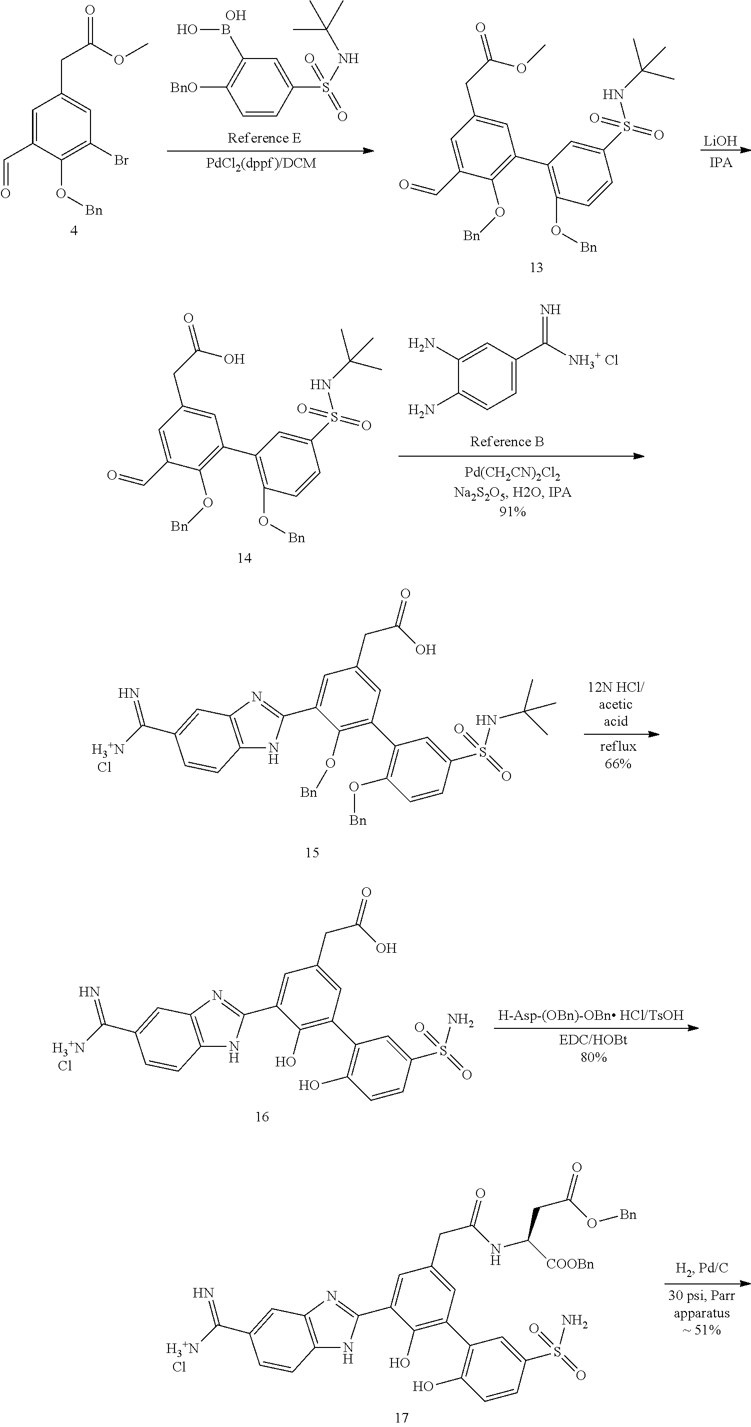

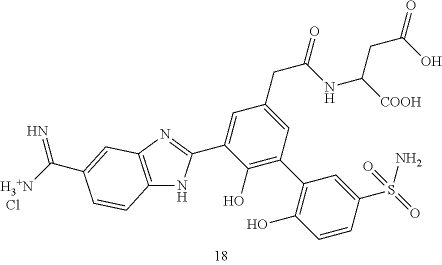

### Example 2

### Synthesis of (R)-2-{2-[5-(5-carbamimidoyl-1H-benzimidazol-2-yl)-6,2′-dihydroxy-5′-sulfamoylbiphenyl-3-yl]acetylamino}succinic acid (20)

The R-enantiomer (20) was synthesised as depicted in scheme 2 starting from compound (16) and exchanging (L)-H-Asp-(OBn)-OBn).TsOH with the corresponding (D)-H-Asp-(OBn)-OBn.TsOH (Bachem) but applying similar reaction conditions used for synthesis of compound (17).

Synthesis of Compound (19):

Compound (16) (65.2 g), was dissolved in DMF (650 g), and the solution was cooled to −5° C. (D)-Asp(OBn)₂*p-TsOH (64.1 g, 1.05 eq.) and N-methyl-morpholine (51.1 g, 4.0 eq.) was added. The suspension was stirred at −5° C. until a solution was obtained, and HATU (50.0 g, 1.05 eq.) was added. The reaction mixture was stirred for 1 hour at −5° C. and transferred to a mixture of MeCN (425 g), 2-propanol (628 g) and demineralized water (3117 g) at 45° C. The clear solution was seeded, stirred for 3 hrs at 35-40° C., and cooled to 10° C. The suspension was stirred overnight at 10° C. and filtered. The filter cake was washed with demineralized water (355 g) and dried in vacuum at 25° C. to obtain 72.1 g of compound (19) as R-isomer.

Yield: 73.8%

Purity (HPLC@230 nm): 89.3%

Synthesis of Compound (20):

Compound (19) (69.9 g) was suspended in acetic acid (1417 g) and demineralized water (720 g). The suspension was heated to 45° C., catalyst (1.97 g, 20% Pd(OH)₂) was added, and the mixture was hydrogenated at 1150-1200 mbar for 1 hour to form a clear solution. The catalyst was filtered off, and the filtrate was concentrated to dryness. The crude product was suspended in acetic acid (1084 g) and water (824 g), and heated to 80° C. to form a solution. Demineralized water (3275 g) was slowly added. After 1.0 L was added, the mixture was seeded (T=62° C.), and the remaining water was added, while keeping the temperature at 55° C. The suspension was cooled to 0° over 6 hrs and stirred overnight at 0° C. The product was isolated by filtration, the filter cake was washed with demineralized water (314 g) and dried in vacuum at 25° C. to obtain 39.3 g of compound (20) (R-isomer) as a yellow crystalline solid.

Yield: 73.1%

Purity (HPLC@230 nm): 97.7%

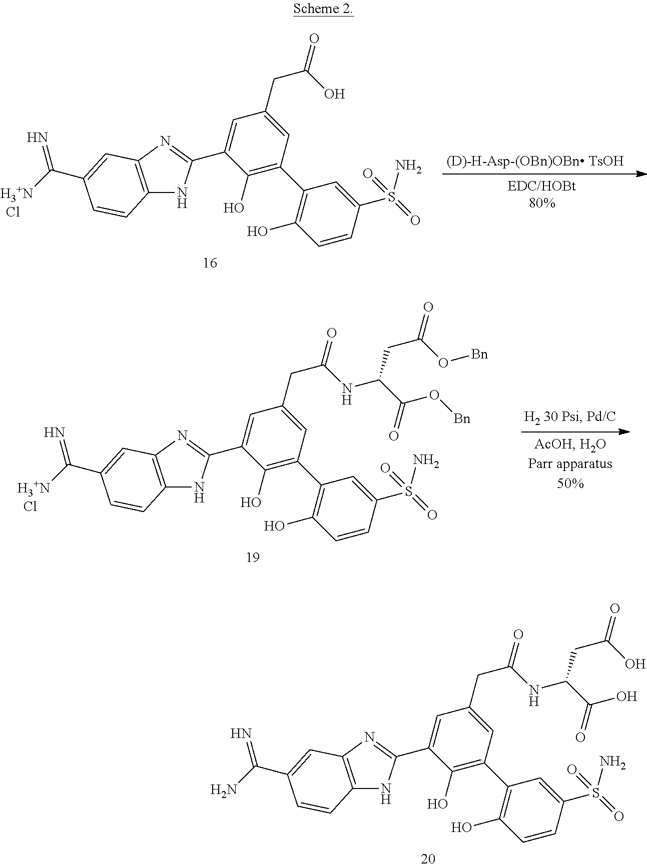

### Example 3

### Degradation of FVIIa

Real time stability of different liquid formulations including an active site stabilizing agent have been tested in cartridges stored in ambient humidity and darkness at 5° C., 25° C. and 30° C. The real time stability study shows that it is possible to achieve a stable liquid rFVIIa or liquid rFVIIa analogue product when stored at 5° C. and during short time storage at 25° C. or 30° C. The heavy chain fragmentation is inhibited effectively by the active site stabilizing agent. No increase in heavy chain fragments are observed at 5° C. and only a very slight increase is observed during 2 months at 25° C. or 30° C. No oxidation is observed at 5° C. and limited oxidation is observed at 25° C. or 30° C., when adequately amounts of an antioxidant is added to the formulation. Deamidation of rFVIIa is observed, and observed to increase with increasing pH and temperature. Stability studies shows that the potency is not influenced by the increase in the level of deamidated forms.

The following seven compositions were made:

A: 1 mg/mL (=20 μM) rFVIIa, 30 μM active site stabilizing agent, 128.3 mM NaCl, 8 mM CaCl₂, 2H₂O, 10 mM Histidine, 3.4 mM Methionine, 10 mM glycylglycine, 0.07 mg/mL Tween80, at pH 6.7.

B: 4.5 mg/mL (=20 μM) rFVIIa, 30 μM active site stabilizing agent, 128.3 mM NaCl, 8.7 mM CaCl2, 2H2O, 10 mM Histidine, 3.4 mM Methionine, 10 mM glycylglycine, 0.07 mg/mL Tween80, at pH 6.7.

C: 1 mg/mL (=20 μM) rFVIIa, 40 μM active site stabilizing agent, 128.3 mM NaCl, 8 mM CaCl₂, 2H₂O, 10 mM Histidine, 6.8 mM Methionine, 10 mM glycylglycine, 0.5 mg/ml Poloxamer 188, pH 6.7.

D: 1 mg/mL (=20 μM) SF-rFVIIa, 30 μM active site stabilizing agent, 128.3 mM NaCl, 8 mM CaCl₂, 2H₂O, 10 mM Histidine, 3.4 mM Methionine, 10 mM glycylglycine, 0.07 mg/mL Tween80, at pH 6.7.

E: 1 mg/mL (=20 μM) SF-rFVIIa, 128.3 mM NaCl, 10 mM CaCl₂, 2H₂O, 10 mM Histidine, 3.4 mM Methionine, 10 mM glycylglycine, 0.07 mg/mL Tween80, at pH 6.5.

F: 1 mg/mL (=20 μM) rFVIIa analogue (V158D/E296V/M298Q-FVIIa), 25 μM active site stabilizing agent, 128.3 mM NaCl, 10 mM CaCl₂, 2H₂O, 10 mM Histidine, 3.4 mM Methionine, 10 mM glycylglycine, 0.07 mg/mL Tween80, pH 6.5.

G: 1 mg/mL (=20 μM) rFVIIa analogue (V158D/E296V/M298Q-FVIIa), 50 μM active site stabilizing agent, 128.3 mM NaCl, 10 mM CaCl₂, 2H₂O, 10 mM Histidine, 3.4 mM Methionine, 10 mM glycylglycine, 0.07 mg/mL Tween80, pH 6.5.

The compositions were subjected to storage at 5° C., 25° C. and 30° C. At selected intervals samples were taken out of storage and tested for Heavy Chain fragmentation (denoted “HC fragments”) and oxidised forms as described in Assay 2, for aggregation (denoted as “Dimers/Oligomers” as described in Assay 3, and for deamidated forms as described in Assay 4.

TABLE 1
Heavy chain fragments [%]

| | Storage time in | | | | Storage time in | | | | Storage time in | | |
|---|---|---|---|---|---|---|---|---|---|---|---|
| Formu- | months at 5° C. | | | | months at 25° C. | | | | months at 30° C. | | |
| lation | 0 | 1 | 3 | 6 | 0.5 | 1 | 2 | 3 | 0.5 | 1 | 2 |
| A | 10.3 | 10.4 | 10.4 | 10.0 | 10.5 | 10.4 | 10.8 | 10.9 | — | — | — |
| B | 11.9 | 12.2 | 12.3 | 11.6 | 12.4 | 12.2 | 12.6 | 12.8 | — | — | — |
| C | 6.9 | 6.8 | 6.9 | 6.6 | 6.9 | 6.9 | 6.9 | 7.2 | — | 7.1 | 7.2 |
| D | 5.2 | 5.3 | 5.5 | 5.5 | 5.7 | 5.9 | 8.2 | 10.5 | — | — | — |
| E | 6.7 | 19.6 | — | — | 19.8 | 34.8 | — | — | — | — | — |
| F | 3.5 | — | 3.5 | — | 3.5 | 4 | — | 4.8 | 3.7 | 4.5 | — |
| G | 3.6 | — | 3.4 | — | 3.3 | 3.6 | 3.4 | 3.5 | 3.3 | 3.6 | 3.5 |

TABLE 2
Oxidised forms [%]

| | Storage time in | | | | Storage time in | | | | Storage time in | | |
|---|---|---|---|---|---|---|---|---|---|---|---|
| Formu- | months at 5° C. | | | | months at 25° C. | | | | months at 30° C. | | |
| lation | 0 | 1 | 3 | 6 | 0.5 | 1 | 2 | 3 | 0.5 | 1 | 2 |
| A | 1.1 | 1.3 | 1.6 | 1.4 | 1.4 | 1.5 | 2.2 | 2.8 | — | — | — |
| B | 1.1 | 1.4 | 1.7 | 1.6 | 1.8 | 1.7 | 2.3 | 2.9 | — | — | — |
| C | 1.9 | 1.8 | 2.0 | 1.9 | 2.2 | 2.1 | 2.6 | 2.8 | — | 2.5 | 3.2 |
| D | 1.1 | 1.3 | 1.6 | 1.5 | 1.6 | 1.5 | 2.1 | 2.8 | — | — | — |
| E | 1.3 | 1.1 | — | — | 2.1 | 2.5 | — | — | — | — | — |
| F | 2.5 | — | 2.4 | — | 2.5 | 3.1 | — | 3.7 | 2.8 | 3.7 | — |
| G | 2.6 | — | 2.4 | — | 2.6 | 3.3 | 3.2 | 3.8 | 2.7 | 3.6 | 4.1 |

TABLE 3
Dimers/Oligomers [%]

| | Storage time in | | | | Storage time in | | | | Storage time in | | |
|---|---|---|---|---|---|---|---|---|---|---|---|
| Formu- | months at 5° C. | | | | months at 25° C. | | | | months at 30° C. | | |
| lation | 0 | 1 | 3 | 6 | 0.5 | 1 | 2 | 3 | 0.5 | 1 | 2 |
| A | 0.5 | 0.5 | 0.5 | 0.4 | 0.5 | 0.4 | 0.4 | 0.5 | — | — | — |
| B | 1.2 | 1.1 | 1.2 | 1.0 | 1.3 | 0.9 | 0.9 | 1.2 | — | — | — |
| C | 8.7 | 8.0 | 7.7 | 7.3 | 0.4 | 0.4 | 0.4 | 0.4 | — | 0.5 | 0.5 |
| D | 0.4 | 0.4 | 0.4 | 0.2 | 0.3 | 0.2 | 0.2 | 0.2 | — | — | — |
| E | 1.3 | 1.4 | — | — | 1.1 | 1.3 | — | — | — | — | — |
| F | 2.2 | — | 2.2 | — | 2.0 | 1.8 | — | 2.1 | 1.9 | 1.8 | — |
| G | 2.0 | — | 2.4 | — | 2.0 | 1.9 | 2.2 | 2.1 | 1.9 | 1.9 | 2.2 |

TABLE 4
Deamidation [%]

| | Storage time in months at | | Storage time in months at | |
|---|---|---|---|---|
| | 5° C. | | 25° C. | |
| Formulation | 0 | 3 | 1 | 3 |
| A | 5 | 6 | 11 | 25 |
| B | 5 | 6 | 11 | 26 |
| C | 4 | 6 | 12 | 25 |
| D | 4 | 7 | 10 | 28 |
| E | 4 | — | 10 | — |
| F | — | — | — | |
| G | — | — | — | |

### Example 4

### Potency of FVIIa

Seven formulations, A, B, C, D, E, F and G composed as described in Example 3, were subjected to storage at 40° C. for 14 days. Each day, samples were taken out of storage and tested for potency (FVIIa activity). Potency was shown by a clot-assay (as described in Assay no 1).

TABLE 5
Potency [IU/ml]

| | Storage time in | | | Storage time in | | | Storage time in |
|---|---|---|---|---|---|---|---|
| Formula- | months at 5° C. | | | months at 25° C. | | | months at 30° C. |
| Tion | 0 | 1 | 3 | 1 | 2 | 3 | 3 |
| A | 46530 | — | 52600 | 47300 | 44000 | — | — |
| B | 231806 | — | 252100 | 230900 | 239600 | — | — |
| C | 51200 | 50700 | — | 50400 | — | — | — |
| D | 54585 | — | 59300 | 54400 | — | — | — |
| E | 60755 | — | — | — | — | — | — |
| F | 677819 | — | 710336 | — | — | 712988 | 744392 |
| G | 689020 | — | — | — | — | — | — |

The experiment shows that FVIIa activity (potency) is maintained in the presence of the active site stabilizing agent with Formula I.

### Example 5

### Degradation of rFVIIa in the Presence of the Active Site Stabilizing Agent with Formula II (R-Isomer)

Accelerated stability of rFVIIa in different liquid formulations including the active site stabilizing agent set forth in Formula II (R-isomer) was tested at 25° C. and 40° C., respectively. The tests were conducted in 1 mL HPLC vials stored at ambient humidity and darkness.

The following compositions were made:
H. 1 mg/mL SF-rFVIIa, 50 μM active site stabilizing agent (R-isomer), 10 mM CaCl₂, 2H₂O, 128.3 mM NaCl, 10 mM glycylglycine, 3.4 mM L-methionine, 10 mM L-histidine, 0.07 mg/mL tween 80, 0.5% (v/v) dimethylsulfoxide, pH 6.0
I. 1 mg/mL SF-FVIIa, 150 μM active site stabilizing agent (R-isomer), 10 mM CaCl₂, 2H₂O, 128.3 mM NaCl, 10 mM glycylglycine, 3.4 mM L-methionine, 10 mM L-histidine, 0.07 mg/mL tween 80, 0.5% (v/v) dimethylsulfoxide, pH 6.0

Composition H was subjected to storage at 25° C. and 40° C., while composition I was subjected to storage at 40° C. Samples were taken out of storage at selected intervals (Days 0, 1, 7 and 14) and tested for heavy chain fragmentation and oxidation as described in Assay 2 and for aggregation as described in Assay 3.

TABLE 6
Heavy chain fragments [%]

| | | |
|---|---|---|
| | Storage time in | Storage time in |
| Formu- | days at 25° C. | days at 40° C. |

| lation | 0 | 1 | 7 | 14 | 0 | 1 | 7 | 14 |
|---|---|---|---|---|---|---|---|---|
| H | 6.3 | 6.3 | 6.4 | 6.4 | 6.0 | 6.3 | 6.0 | 5.4 |
| I | — | — | — | — | 6.3 | 6.2 | 6.4 | 5.1 |

TABLE 7
Oxidised forms [%]

| | | |
|---|---|---|
| | Storage time in | Storage time in |
| Formu- | days at 25° C. | days at 40° C. |

| lation | 0 | 1 | 7 | 14 | 0 | 1 | 7 | 14 |
|---|---|---|---|---|---|---|---|---|
| H | 2.1 | 1.7 | 1.7 | 1.6 | 1.6 | 2.0 | 2.6 | 3.6 |
| I | — | — | — | — | 2.7 | 2.8 | 4.4 | 4.5 |

TABLE 8
Dimers/Oligomers[%]

| | | |
|---|---|---|
| | Storage time in | Storage time in |
| Formu- | days at 25° C. | days at 40° C. |

| lation | 0 | 1 | 7 | 14 | 0 | 1 | 7 | 14 |
|---|---|---|---|---|---|---|---|---|
| H | 0.1 | 0.1 | 0.1 | 0.1 | 0.1 | 0.2 | 0.3 | 0.3 |
| I | — | — | — | — | 0.2 | 0.2 | — | 0.2 |

The study showed that it was possible to achieve a stable liquid rFVIIa product using the active site stabilizing agent excipient during short time storage at 25° C. or 40° C. No increase in heavy chain fragments or aggregation was observed during 14 days at 25° C. or 40° C. No oxidation was observed at 25° C. but oxidation was observed at 40° C., when an inadequately amount of an antioxidant was added to the formulation.

### Example 6

### Degradation of rFVIIa in the Presence of Active Site Stabilizing Agent

Accelerated stability of rFVIIa in different liquid formulations including S-2-[3-(4-Carbamimidoylphenyl)ureido]-N-[1-(3-methoxyphenyl)-ethyl]-acetamide (formula A) (designated “008”) was tested at 25° C. and 40° C., respectively. The tests were conducted in 1 mL HPLC vials stored at ambient humidity and darkness.

### S-2-[3-(4-Carbamimidoylphenyl)ureido]-N-[1-(3-methoxyphenyl)-ethyl]-acetamide

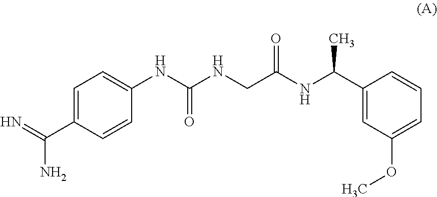 [SMILES: COc1cccc([C@H](C)NC(=O)CNC(=O)Nc2ccc(C(=N)N)cc2)c1]

The following compositions were made:
J. 1 mg/mL SF-rFVIIa, 150 μM S-2-[3-(4-Carbamimidoylphenyl)ureido]-N-[1-(3-methoxyphenyl)-ethyl]-acetamide, 10 mM CaCl₂, 2H₂O, 128.3 mM NaCl, 10 mM glycylglycine, 3.4 mM L-methionine, 10 mM L-histidine, 0.07 mg/mL tween 80, 0.5% (v/v) dimethylsulfoxide, pH 6.0
K. 1 mg/mL SF-rFVIIa, 500 μM S-2-[3-(4-Carbamimidoylphenyl)ureido]-N-[1-(3-methoxyphenyl)-ethyl]-acetamide, 10 mM CaCl₂, 2H₂O, 128.3 mM NaCl, 10 mM glycylglycine, 3.4 mM L-methionine, 10 mM L-histidine, 0.07 mg/mL tween 80, 0.5% (v/v) dimethylsulfoxide, pH 6.0

Composition J and K were subjected to storage at 25° C. and 40° C. Samples were taken out of storage at selected intervals (Days 0, 1, 7 and 14) and tested for heavy chain fragmentation and oxidation as described in assay 2. The appearance of not previously identified degradation products in assay 2 (=RP-HPLC) was apparent upon storage at 40° C. Aggregation was analysed as described in assay 3.

TABLE 9
Heavy chain fragments [%]

| | | |
|---|---|---|
| | Storage time in | Storage time in |
| Formu- | days at 25° C. | days at 40° C. |

| lation | 0 | 1 | 7 | 14 | 0 | 1 | 7 | 14 |
|---|---|---|---|---|---|---|---|---|
| J | 6.3 | 6.5 | 7.1 | 8.0 | 6.4 | 6.2 | 7.6 | 8.5 |
| K | 6.1 | 6.6 | 6.8 | 7.6 | 6.8 | 6.6 | 7.4 | 8.7 |

TABLE 10
Oxidised forms [%]

| | | |
|---|---|---|
| | Storage time in | Storage time in |
| Formu- | days at 25° C. | days at 40° C. |

| lation | 0 | 1 | 7 | 14 | 0 | 1 | 7 | 14 |
|---|---|---|---|---|---|---|---|---|
| J | 1.7 | 1.5 | 2.3 | 1.8 | 1.5 | 2.1 | 3.1 | 4.2 |
| K | 1.7 | 1.4 | 1.4 | 1.9 | 1.7 | 2.5 | 3.4 | 4.5 |

TABLE 11
Unidentified degradation products [%]

| | | |
|---|---|---|
| | Storage time in | Storage time in |
| Formu- | days at 25° C. | days at 40° C. |

| lation | 0 | 1 | 7 | 14 | 0 | 1 | 7 | 14 |
|---|---|---|---|---|---|---|---|---|
| J | 1.4 | 1.5 | 2.0 | 2.0 | 1.7 | 2.2 | 5.7 | 8.9 |
| K | 1.7 | 1.5 | 1.3 | 1.4 | 1.2 | 2.1 | 5.1 | 7.0 |

TABLE 12
Dimers/Oligomers[%]

| | | |
|---|---|---|
| | Storage time in | Storage time in |
| Formu- | days at 25° C. | days at 40° C. |

| lation | 0 | 1 | 7 | 14 | 0 | 1 | 7 | 14 |
|---|---|---|---|---|---|---|---|---|
| J | 0.4 | 0.4 | 0.4 | 0.4 | 0.4 | 1.2 | 3.3 | 4.3 |
| K | 0.4 | 0.2 | 0.2 | 0.3 | 0.2 | 0.9 | 3.7 | 10.0 |

The study showed that the liquid rFVIIa product containing the compound set forth in formula II (R-form) achieved a better stability compared with the liquid rFVIIa product using the Formula A excipient in the concentration range from 150 μM to 500 μM at condition J and K and during short time storage at 25° C. or 40° C. An increase in rFVIIa heavy chain fragments, unidentified degradation products, oxidised forms and aggregation was observed during 14 days at 40° C. The increase in all degradation products except heavy chain fragments was minor at 25° C.

### Example 7

### Bioactivity of rFVIIa in the Presence of Active Site Stabilizer

The biological in vivo efficacy and potency of recombinant factor VIIa (rFVIIa) co-formulated with the active site stabilizing agent with Formula I in the molar ratio 1:1.75 compared to rFVIIa at the dose 1.25; 2.5; 5; 10 and 12.5 mg/kg was study in tail bleeding in FVIII knock out (F8-KO) mice (Bi L, Sarkar R, Naas T, Lawler A M, Pain J, Shumaker S L et al. Further characterization of factor VIII-deficient mice created by gene targeting: RNA and protein studies. Blood 1996; 88:3446-).

Tail bleeding was initiated in Isofluran anesthetized F8-KO mice by transection of 4 mm of the tip of the tail 5 min after dosing rFVIIa, rFVIIa:active site stabilizing agent (1:1.75) or vehicle iv in a tail vein in the mice. Bleeding time and blood loss was measured for a 30 minutes period in 37° C. saline as described elsewhere (Elm T; Karpf D M; Øvlisen K; Pelzer H; Ezban M; Kjalke M; Tranholm M. Pharmacokinetics and pharmacodynamics of a new recombinant FVIII (N8) in haemophilia A mice. Haemophilia, 2012; 18 (1), 139-145). The blood loss ED50 was calculated to 2.12 mg/kg (95% CI 1.28-3.53) for rFVIIa and 2.05 mg/kg (95% CI 0.92-4.53) for rFVIIa:active site stabilizing agent (1:1.75), respectively, p=0.94. The bleeding time vs dose of rFVIIa and rFVIIa:active site stabilizing agent (1:1.75) show very similar dose response curves.

In conclusions, there was no significant difference in dose response between the rFVIIa and rFVIIa co-formulated with the active site stabilizing agent in acute tail bleeding in F8-KO mice.

### Example 8

### Bioactivity of SF-rFVIIa in the Presence of Active Site Stabilizer

The in vivo effect of serum free recombinant FVIIa (SF-rFVIIa) and SF-rFVIIa co-formulated with the active site stabilizing agent set forth in Formula I in the molar ratio 1:2.5 was studied with the same design in the tail bleeding model in F8-KO mice at the concentration 1; 2.5; 5; 10 and 15 mg/kg. The blood loss ED₅₀ was in this study calculated to 2.1 mg/kg for SF-rFVIIa and 2.6 mg/kg for SF-rFVIIa:active site stabilizing agent (1:2.5), respectively, p=0.53 (data not shown). The bleeding time versus dose and the blood loss and bleeding time vs the exposure of SF-FVIIa and SF-rFVIIa:active site stabilizing agent show very similar dose response curves. The exposure mean values of SF-rFVIIa both as measured by ELISA and clot activity indicated significant increased exposure to SF-rFVIIa when co-formulated with the active site stabilizing agent (Two way ANOVA P<0.01). The antigen concentrations measured in plasma after the highest dose (15 mg/kg) were 1168±50 nM and 1365±152 nM for SF-rFVIIa and SF-rFVIIa with the active site stabilizing agent (P═NS), respectively. At the same dose the clot activity was 1195 nM for SF-rFVIIa and 1735 nM for SF-rFVIIa when co-formulated with active site stabilizing agent (P<0.001). Despite this increase in exposure no statistically significant impact of active site stabilizing agent on EC₅₀ estimates were identified.

In conclusion, comparable dose response relationships were demonstrated for SF-rFVIIa alone or co-formulated with active site stabilizing agent (1:2.5) in a tail bleeding model in hemophilia A mice. Normalization of the bleeding was observed at 15 mg/kg SF-rFVIIa alone and co-formulated with active site stabilizing agent. Increased exposure to SF-rFVIIa (ELISA and clot activity) was observed when SF-rFVIIa was co-administered with active site stabilizing agent. Despite the higher plasma levels no significant differences in EC₅₀'s were detected.

### Example 9

### Bioactivity of a FVIIa Sequence Variant, V158D/E296V/M298Q-FVIIa in the Presence of Active Site Stabilizer

In the same tail bleeding model in F8-KO mice we studied the effect of using the S or R form of the active site stabilizing agent when co-formulated with SF-rFVIIa and the effect of a rFVIIa variant (V158D/E296V/M298Q-FVIIa) (Vatreptacog Alfa) dosed alone or in combination with the active site stabilizing agent set forth in Formula I (1:2.5) (Table 13).

Vatreptacog Alfa is a FVIIa sequence variant, V158D/E296V/M298Q-FVII (numbering referring to sequence of human wild-type FVIIa, SEQ ID NO:1), wherein three amino acids of the wild-type human sequence have been replaced.

The blood loss were significantly longer in vehicle-dosed F8-KO mice compared to normal C57BL mice (p<0.001). The administrations of 10 mg/kg of SF-rFVIIa or SF-rFVIIa with the active site stabilizing agent set forth in Formula I (S-form) in the ratios of 1:1 or 1:2.5 and active site stabilizing agent with formula II (R-form) (1:1) significantly reduced the blood loss in F8-KO mice (p<0.001 compared to F8-KO control mice). The administration of 3 mg/kg of Vatreptacog Alfa or Vatreptacog Alfa:active site stabilizing agent (1:2.5) significantly reduced the blood loss in F8-KO mice (p<0.001 compared to F8-KO control mice). Blood losses from the compound dosed groups did not significant differ from that of the vehicle treated C57BL control group.

In conclusion, SF-FVIIa and Vatreptacog Alfa alone or co-formulated with active site stabilizing agent up to a molar ratio of 1:2.5 normalized the blood loss in F8-KO mice. No significant difference was found between the R and S form of active site stabilizing agent.

TABLE 13
In vivo tail bleeding as blood loss
(nmol haemoglobin) in F8-KO mice.

| | Dose | | | |
|---|---|---|---|---|
| Groups | mg/kg | n | mean | sem |
| F8-KO control | 0 | 8 | 5571 | 504 |
| C57BL control | 0 | 8 | 833 | 409 |
| SF-FVIIa 10 mg/kg | 10 | 10 | 631 | 158 |
| SF-rFVIIa:agent with Formula I | 10 | 8 | 535 | 202 |
| (S-form) 1:1 | | | | |
| SF-rFVIIa:agent with Formula I | 10 | 8 | 801 | 236 |
| (S-form) 1:2.5 | | | | |
| SF-rFVIIa:agent with Formula II | 10 | 8 | 1243 | 511 |
| (R-form) 1:1 | | | | |
| Vatreptacog Alfa | 3 | 8 | 1207 | 500 |
| Vatreptacog Alfa:agent with | 3 | 8 | 1009 | 363 |
| Formula I (S-form) 1:2.5 | | | | |

I.v. injections were given 5 minutes before induction of bleeding by cutting a 4 mm tip of the tail. All groups are significant different compared to F8-KO mice (p<0.0001), no significant different were found between the dosing groups or C57BL control mice (One way ANOVA).

In conclusion, these experiment shows that the active site stabilizing agent set forth Formulas I and II (in the S or R form) does not impair the biological activity of rFVIIa, SF-FVIIa or Vatreptacog Alfa in a tail bleeding model in F8-KO mice.

### Example 10

### Binding of Active Site Stabilizing Agent to rFVIIa Polypeptides

All proteases were dialyzed extensively in binding buffer: 10 mM HEPES pH 7.4, 150 mM NaCl, 0.005% v/v Surfactant P20, 5 mM CaCl₂. All binding experiments were carried out in binding buffer unless otherwise indicated. The active site stabilizing agent set forth Formula I was dissolved in 50 mM Tris pH 8.0 to a final concentration of 9 mM giving a yellow colour. Isothermal titration calorimetry (iTC₂₀₀, from GE healthcare) was chosen as the method of choice for determining binding parameters. Each iTC₂₀₀ run involved filling the cell with the protease (approximately 200 μL) and the syringe with the active site stabilizing agent (approximately 40 μL). Temperature was set as required and the protease was allowed to equilibrate under given experimental conditions (approximately 10 minutes). Typically 17-20 injections (of 2-2.5 μL) of active site stabilizing agent into the cell, containing protease, were performed. The first injection was always of 0.2 μL and was discarded from the final data analysis. Stirring speed was set between 700-1000 rpm. Filter period for data collection was 5 sec with a high feedback mode setting. Each titration was spaced by 120 sec. Raw data was processed to set the baseline, integrate each peak to get a final isotherm. This isotherm was fit to a single-site model to yield K_{d}, stoichiometry (n), ΔH, and ΔS values to complete characterization of binding of active site stabilizing agents to a protease. Each binding experiment was repeated at least twice. Tables 14, 15 and 16 summarizes binding of active site binding agents to SF-FVIIa and Vatreptacog alfa under varying solution conditions as described below.

TABLE 14
Active site stabilizing agent binding, K_{d}

| | Formula A | Formula II | Formula I |
|---|---|---|---|
| SF-rFVIIa | 1.78 μM | 12 nM | 20 nM |

Table 14: Summary of dissociation constant, K_{d}, for binding of different active site stabilizing agents to SF-FVIIa using iTC200. Measurements were made in binding buffer and 20° C. The Formula A excipient bound to SF-FVIIa with an affinity of 1.78 uM. The active site stabilizing agent with formula II (R-form) bound to SF-rFVIIa with an affinity of 12 nM, and of the active site stabilizing agent with formula I (S-form) bound to SF-rFVIIa with an affinity of 20 nM.

TABLE 15
Binding of the active site stabilizing
agent with formula I (S-form), K_{d}

| | | |
|---|---|---|
| Temperature | 20° C. | 37° C. |

| | | | |
|---|---|---|---|
| SF-rFVIIa | 20 | nM | 0.334 uM |
| rFVIIa | 12.9 | nM | 0.296 uM |
| V158D/E296V/M298Q-FVIIa | 2.18 | nM | 0.046 uM |

Table 15: Summary of dissociation constant, K_{d}, for binding of the active site stabilizing agent with formula I (S-form) to SF-rFVIIa, rFVIIa and V158D/E296V/M298Q-FVIIa using iTC₂₀₀. Measurements were made in binding buffer at different temperatures (20° C. and 37° C.) as indicated in the table. It was observed that binding of the active site stabilizing agent with formula I (S-form) to SF-rFVIIa, rFVIIa, and Vatreptacog alfa was weaker at higher temperature. The fold difference in binding at 20° C. and 37° C. was 17-fold, 23-fold, and 21-fold for SF-FVIIa, rFVIIa, and V158D/E296V/M298Q-FVIIa, respectively.

TABLE 16
Binding of the active site stabilizing agent
with formula I (S-form), Kd at 20° C.

| pH | 5.5 | | 6.5 | | 7.4 | | 8.5 | |
|---|---|---|---|---|---|---|---|---|
| SF-rFVIIa | 1.56 | uM | 43 | nM | 20 | nM | 0.13 | uM |
| V158D/E296V/M298Q- | 77 | nM | 9 | nM | 2.18 | nM | 6 | nM |
| FVIIa | | | | | | | | |

Table 16. Summary of dissociation constant, K_{d}, for binding of the of the active site stabilizing agent with formula I (S-form) to SF-rFVIIa and V158D/E296V/M298Q-FVIIa using iTC₂₀₀. Measurements were made in binding buffer but with varying pH. Both proteases displayed highest affinity for of the active site stabilizing agent with formula I (S-form) at pH 7-7.5. Compared to SF-rFVIIa, V158D/E296V/M298Q-FVIIa displayed less dependence on pH.

### Example 11

### Active site stabilization of FVIIa by (S)-2-{2-[5-(5-carbamimidoyl-1H-benzoimidazol-2-yl)-6,2′-dihydroxy-5′-sulfamoyl-biphenyl-3-yl]acetylamino}-succinic acid described by X-ray crystallography

Materials

The Gla-domain truncated form of human FVIIa (amino acid residues 46-406 of SEQ ID NO:1) in a buffer consisting of 10 mM 2-Amino-2-hydroxymethyl-propane-1,3-diol, 100 mM NaCl, 15 mM CaCl₂ pH 7.4 at a protein concentration of 7 mg/mL and a (S)-2-{2-[5-(5-carbamimidoyl-1H-benzoimidazol-2-yl)-6,2′-dihydroxy-5′-sulfamoyl-biphenyl-3-yl]acetylamino}-succinic acid concentration of more than 140 μM.

Methods

Protein Crystallization

The Gla-domain truncated form of FVIIa in complex with (S)-2-{2-[5-(5-carbamimidoyl-1H-benzoimidazol-2-yl)-6,2′-dihydroxy-5′-sulfamoyl-biphenyl-3-yl]acetylamino}-succinic acid was crystallized in a sitting drop vapour diffusion experiment at 20° C. by equilibration of a droplet consisting of 100 nL protein solution and 100 nL reservoir solution against a reservoir solution composed of 15% (w/v) polyethylene glycol 20000, 100 mM 2-[4-(2-hydroxyethyl)piperazin-1-yl]ethanesulfonic acid pH 7.0. Crystals appeared after 2 weeks and continued to grow for additionally 2 weeks.

The crystal and the crystallization drop was covered with 1 μL 4 M trimethylamine N-oxide dihydrate and the crystal was dragged through the trimethylamine N-oxide dihydrate and mounted in a 0.06 mm diameter litholoop (Molecular Dimensions Limited) followed by flash-cooling of the crystal in liquid nitrogen for diffraction analysis.

X-Ray Diffraction Data Collection, Structure Determination and Refinement

Diffraction data were collected at the MX beam line at the Maxlab II synchrotron operated at a wavelength of 1.000 Å, with a crystal to detector distance of 198.15 mm and an oscillation width per frame of 0.5 degree. The raw data images were indexed, integrated and scaled using the mosflm program (Leslie and Powell, NATO Science Series, 245, 41-51 (2007)) and the scala program (Potterton et al., Acta Crystallogr. D59, 1131-1137 (2003)). The space group of the crystal was P2(1)2(1)2(1), with unit cell parameters, a=94.1 Å, b=94.2 Å, c=107.3 Å, α=90°, μ=90°, γ=90°. Data were collected to a resolution of 1.90 Å. The data were twinned with the twin operator (K, H, −L) and a twin fraction of 0.495. The structure was solved by molecular replacement using the Molrep software (Vagin and Teplyakov, J. Appl. Cryst. 30, 1022-1025 (1997)) as implemented in the CCP4i program suite (Potterton et al., Acta Crystallogr. D59, 1131-1137 (2003)). The search model was the structure of the human FVIIa described by Banner et al. (Nature 380, 41-46 (1996)). Two copies of Gla-domain truncated FVIIa were located in the asymmetric unit. Structure refinement was carried out using Refmac5 (Murshudov er al., Acta Crystallogr. D53, 240-255 (1997)) from the CCP4i program suite and Coot version 7 (Emsley et al., Acta Crystallogr. D66, 486-501 (2010)) was used for manual structure rebuilding and validation.

### RESULTS AND DISCUSSION

The crystal structure coordinates of the complex between the Gla-domain truncated FVIIa and (S)-2-{2-[5-(5-carbamimidoyl-1H-benzoimidazol-2-yl)-6,2′-dihydroxy-5′-sulfamoyl-biphenyl-3-yl]acetylamino}-succinic acid included amino acid residues L89-R144, I153-P406 (SEQ ID NO: 1) from one copy of the FVIIa molecule in the asymmetric unit and amino acid residues L89-K143, I153-K316, P321-P406 (SEQ ID NO: 1) from the other copy of the FVIIa molecule in the asymmetric unit. The overall R-factor of the refined structure was 18.0% and the free R-factor was 20.6%. The overall correlation coefficient was 0.96 and the diffraction-component precision index, DPI=0.02 Å (Cruickshank, Acta Crystallogr. D55, 583-601 (1999)). The root-mean-square deviation of the bond lengths in the structure from ideal bond lengths=0.0044 Å and the root-mean-square deviation from ideal bond angles=1.1246° (Engh and Huber, Acta Crystallogr. A47, 392-400 (1991)). Amino acid residues displaying intermolecular distances of less than or equal to 4 Å between the Gla-domain truncated FVIIa molecule and the (S)-2-{2-[5-(5-carbamimidoyl-1H-benzoimidazol-2-yl)-6,2′-dihydroxy-5′-sulfamoyl-biphenyl-3-yl]acetylamino}-succinic acid were assigned as (S)-2-{2-[5-(5-carbamimidoyl-1H-benzoimidazol-2-yl)-6,2′-dihydroxy-5′-sulfamoyl-biphenyl-3-yl]acetylamino}-succinic acid interacting amino acid residues (SEQ ID NO: 1). The analysis of intermolecular distances was carried out using the program Contact in the CCP4 program suite (Potterton et al., Acta Crystallogr. D59, 1131-1137 (2003)) and showed the amino acid residues listed in table 17 to comprise the (S)-2-{2-[5-(5-carbamimidoyl-1H-benzoimidazol-2-yl)-6,2′-dihydroxy-5′-sulfamoyl-biphenyl-3-yl]acetylamino}-succinic acid interacting amino acid residues in both FVIIa molecules in the asymmetric unit.

TABLE 17
(S)-2-{2-[5-(5-carbamimidoyl-1H-benzoimidazol-2-yl)-6,2′-dihydroxy-5′-
sulfamoyl-biphenyl-3-yl]acetylamino}-succinic acid interacting amino acid
(positions referring to SEQ ID NO: 1)
H193
C194
D196
K197
D338
S339
C340
K341
S344
V362
S363
W364
G365
G367
C368
G375

At atomic level, the interactions between the active site of FVIIa and (S)-2-{2-[5-(5-carbamimidoyl-1H-benzoimidazol-2-yl)-6,2′-dihydroxy-5′-sulfamoyl-biphenyl-3-yl]acetylamino}-succinic acid involved the atoms listed in table 18.

TABLE 18
asymmetric unit complex 1

| (S)-2-{2-[5-(5- | | | | | |
|---|---|---|---|---|---|
| carbamimidoyl-1H- | | FVIIa | | | |
| benzoimidazol-2- | | amino | | | |
| yl)-6,2′-dihydroxy-5′- | | acid | Amino | | Inter |
| sulfamoyl-biphenyl-3- | | residue | acid | | atomic |
| yl]acetylamino}-succinic | Atom | number | residue | Atom | distance |
| acid atom number | type | and chain | type | name | (Å) |
| C35 | C | 341 H | Lys | NZ | 3.8 |
| C17 | C | 341 H | Lys | NZ | 3.6 |
| O19 | O | 341 H | Lys | CD | 3.6 |
| | | 341 H | Lys | CE | 3.1 |
| | | 341 H | Lys | NZ | 2.5 |
| C14 | C | 341 H | Lys | CG | 3.9 |
| C21 | C | 193 H | His | NE2 | 4.0 |
| C24 | C | 193 H | His | NE2 | 3.8 |
| | | 193 H | His | CD2 | 3.7 |
| C30 | C | 193 H | His | CD2 | 3.7 |
| C28 | C | 193 H | His | O | 3.5 |
| S29 | S | 193 H | His | O | 3.5 |
| | | 197 H | Lys | NZ | 3.9 |
| O33 | O | 197 H | Lys | CD | 3.4 |
| | | 197 H | Lys | CE | 3.4 |
| | | 197 H | Lys | NZ | 2.9 |
| O34 | O | 193 H | His | O | 3.8 |
| | | 196 H | Asp | CB | 4.0 |
| | | 197 H | Lys | N | 3.8 |
| | | 197 H | Lys | CB | 3.7 |
| | | 197 H | Lys | CG | 3.5 |
| | | 197 H | Lys | CD | 3.5 |
| N32 | N | 193 H | His | C | 3.8 |
| | | 193 H | His | O | 2.6 |
| | | 196 H | Asp | CB | 3.4 |
| | | 196 H | Asp | CG | 3.4 |
| | | 196 H | Asp | OD2 | 3.1 |
| C27 | C | 193 H | His | O | 3.7 |
| | | 194 H | Cys | O | 4.0 |
| C25 | C | 193 H | His | NE2 | 3.9 |
| | | 193 H | His | CD2 | 4.0 |
| O31 | O | 341 H | Lys | O | 3.6 |
| C22 | C | 193 H | His | NE2 | 3.5 |
| O23 | O | 341 H | Lys | O | 3.6 |
| | | 344 H | Ser | CB | 3.7 |
| | | 344 H | Ser | OG | 2.8 |
| | | 193 H | His | CE1 | 3.6 |
| | | 193 H | His | NE2 | 2.7 |
| | | 193 H | His | CD2 | 3.6 |
| C13 | C | 341 H | Lys | CG | 3.6 |
| C12 | C | 341 H | Lys | CA | 3.7 |
| | | 341 H | Lys | CG | 3.7 |
| N10 | N | 363 H | Ser | O | 3.6 |
| | | 341 H | Lys | CA | 3.5 |
| | | 344 H | Ser | OG | 2.9 |
| C4 | C | 363 H | Ser | O | 3.6 |
| | | 364 H | Trp | CA | 3.8 |
| | | 340 H | Cys | C | 4.0 |
| | | 341 H | Lys | N | 3.8 |
| | | 341 H | Lys | CA | 3.9 |
| | | 344 H | Ser | OG | 3.5 |
| C3 | C | 362 H | Val | CG1 | 3.7 |
| | | 363 H | Ser | C | 3.6 |
| | | 363 H | Ser | O | 3.5 |
| | | 364 H | Trp | N | 3.7 |
| | | 364 H | Trp | CA | 3.7 |
| | | 340 H | Cys | C | 3.9 |
| | | 340 H | Cys | O | 3.7 |
| | | 344 H | Ser | OG | 3.5 |
| C2 | C | 362 H | Val | CG1 | 3.8 |
| | | 339 H | Ser | OG | 4.0 |
| | | 364 H | Trp | N | 3.8 |
| | | 364 H | Trp | CA | 3.8 |
| | | 364 H | Trp | C | 3.8 |
| | | 364 H | Trp | O | 3.8 |
| | | 339 H | Ser | O | 3.9 |
| N11 | N | 341 H | Lys | CG | 3.9 |
| | | 364 H | Trp | CA | 3.9 |
| C5 | C | 365 H | Gly | N | 3.9 |
| | | 341 H | Lys | N | 4.0 |
| C6 | C | 364 H | Trp | C | 3.6 |
| | | 365 H | Gly | N | 3.4 |
| | | 365 H | Gly | CA | 3.9 |
| | | 367 H | Gly | O | 3.2 |
| | | 365 H | Gly | O | 3.8 |
| C1 | C | 364 H | Trp | CA | 3.9 |
| | | 364 H | Trp | C | 3.5 |
| | | 364 H | Trp | O | 3.5 |
| | | 365 H | Gly | N | 3.7 |
| | | 339 H | Ser | O | 3.5 |
| C7 | C | 338 H | Asp | OD1 | 3.7 |
| | | 338 H | Asp | CG | 3.9 |
| | | 338 H | Asp | OD2 | 3.5 |
| | | 364 H | Trp | C | 3.8 |
| | | 364 H | Trp | O | 3.5 |
| | | 365 H | Gly | N | 4.0 |
| | | 365 H | GLy | CA | 3.9 |
| | | 339 H | Ser | O | 3.0 |
| | | 367 H | Gly | O | 4.0 |
| N9 | N | 338 H | Asp | OD1 | 2.9 |
| | | 339 H | Ser | OG | 3.3 |
| | | 375 H | Gly | CA | 3.2 |
| | | 338 H | Asp | CG | 3.5 |
| | | 338 H | Asp | OD2 | 3.4 |
| | | 364 H | Trp | O | 3.6 |
| | | 339 H | Ser | C | 3.9 |
| | | 339 H | Ser | O | 3.1 |
| N8 | N | 338 H | Asp | OD1 | 3.8 |
| | | 338 H | Asp | CG | 3.6 |
| | | 338 H | Asp | OD2 | 2.8 |
| | | 365 H | Gly | CA | 3.6 |
| | | 339 H | Ser | O | 3.4 |
| | | 367 H | Gly | O | 3.0 |
| | | 368 H | Cys | CA | 3.9 |
| | | 368 H | Cys | CA | 3.9 |

asymmetric unit complex 2

| | | | | | |
|---|---|---|---|---|---|
| O45 | O | 341 M | Lys | NZ | 3.9 |
| C36 | C | 341 M | Lys | CE | 3.8 |
| | | 341 M | Lys | NZ | 3.9 |
| C35 | C | 341 M | Lys | CE | 3.9 |
| N18 | N | 341 M | Lys | CE | 3.7 |
| C17 | C | 341 M | Lys | CE | 3.7 |
| O19 | O | 341 M | Lys | CD | 3.9 |
| | | 341 M | Lys | CE | 3.9 |
| C15 | C | 341 M | Lys | CE | 3.5 |
| C14 | C | 341 M | Lys | CE | 3.6 |
| C20 | C | 341 M | Lys | CE | 3.6 |
| C21 | C | 193 M | His | NE2 | 4.0 |
| | | 341 M | Lys | CE | 3.7 |
| C24 | C | 193 M | His | NE2 | 3.9 |
| | | 193 M | His | CD2 | 3.7 |
| C30 | C | 193 M | His | CD2 | 3.9 |
| C28 | C | 193 M | His | O | 3.8 |
| S29 | S | 193 M | His | O | 3.8 |
| O33 | O | 197 M | Lys | CG | 3.8 |
| | | 197 M | Lys | CD | 3.5 |
| | | 197 M | Lys | CE | 3.9 |
| O34 | O | 197 M | Lys | CG | 3.9 |
| | | 197 M | Lys | N | 3.5 |
| | | 197 M | Lys | CB | 3.9 |
| | | 193 M | His | O | 3.3 |
| | | 196 M | Asp | CB | 3.6 |
| | | 194 M | Cys | O | 4.0 |
| | | 196 M | Asp | N | 3.7 |
| | | 196 M | Asp | CA | 4.0 |
| N32 | N | 193 M | His | O | 3.7 |
| | | 196 M | Asp | CB | 3.6 |
| C27 | C | 193 M | His | O | 4.0 |
| C25 | C | 193 M | His | NE2 | 3.9 |
| | | 193 M | His | CD2 | 4.0 |
| O31 | O | 193 M | His | NE2 | 3.9 |
| | | 341 M | Lys | O | 3.5 |
| | | 344 M | Ser | OG | 4.0 |
| C22 | C | 193 M | His | NE2 | 3.5 |
| | | 193 M | His | CD2 | 4.0 |
| | | 341 M | Lys | CE | 3.8 |
| O23 | O | 193 M | His | CE1 | 3.6 |
| | | 193 M | His | NE2 | 2.6 |
| | | 193 M | His | CD2 | 3.5 |
| | | 341 M | Lys | O | 3.7 |
| | | 344 M | Ser | CB | 3.7 |
| | | 344 M | Ser | OG | 2.9 |
| C13 | C | 341 M | Lys | CG | 3.7 |
| | | 341 M | Lys | CE | 3.8 |
| C12 | C | 341 M | Lys | CG | 3.6 |
| | | 341 M | Lys | CA | 3.9 |
| N10 | N | 363 M | Ser | O | 3.5 |
| | | 341 M | Lys | CA | 3.6 |
| | | 344 M | Ser | OG | 2.9 |
| C4 | C | 363 M | Ser | O | 3.5 |
| | | 364 M | Trp | CA | 3.8 |
| | | 340 M | Cys | O | 4.0 |
| | | 341 M | Lys | N | 3.9 |
| | | 341 M | Lys | CA | 3.8 |
| | | 344 M | Ser | OG | 3.5 |
| C3 | C | 363 M | Ser | C | 3.7 |
| | | 363 M | Ser | O | 3.5 |
| | | 364 M | Trp | N | 3.8 |
| | | 364 M | Trp | CA | 3.8 |
| | | 340 M | Cys | C | 3.9 |
| | | 340 M | Cys | O | 3.5 |
| | | 344 M | Ser | OG | 3.5 |
| | | 362 M | Val | CG1 | 3.8 |
| C2 | C | 364 M | Trp | C | 3.9 |
| | | 364 M | Trp | O | 3.9 |
| | | 364 M | Trp | N | 4.0 |
| | | 364 M | Trp | CA | 3.9 |
| | | 340 M | Cys | C | 4.0 |
| | | 340 M | Cys | O | 3.8 |
| | | 339 M | Ser | OG | 3.9 |
| | | 362 M | Val | CG1 | 3.8 |
| N11 | N | 341 M | Lys | CG | 3.7 |
| C5 | C | 365 M | Gly | N | 3.8 |
| | | 364 M | Trp | C | 4.0 |
| | | 364 M | Trp | CA | 3.8 |
| C6 | C | 365 M | Gly | N | 3.3 |
| | | 364 M | Trp | C | 3.6 |
| | | 365 M | Gly | CA | 3.8 |
| | | 364 M | Trp | CA | 4.0 |
| | | 367 M | Gly | O | 3.3 |
| C1 | C | 365 M | Gly | N | 3.7 |
| | | 364 M | Trp | C | 3.5 |
| | | 364 M | Trp | O | 3.7 |
| | | 364 M | Trp | CA | 4.0 |
| | | 339 M | Ser | O | 3.7 |
| C7 | C | 364 M | Trp | C | 3.9 |
| | | 364 M | Trp | O | 3.7 |
| | | 365 M | Gly | CA | 4.0 |
| | | 339 M | Ser | O | 3.1 |
| | | 367 M | Gly | O | 3.8 |
| | | 338 M | Asp | CG | 3.9 |
| | | 338 M | Asp | OD1 | 3.7 |
| | | 338 M | Asp | OD2 | 3.5 |
| | | 339 M | Ser | C | 4.0 |
| N9 | N | 364 M | Trp | O | 3.7 |
| | | 339 M | Ser | O | 3.2 |
| | | 338 M | Asp | CG | 3.6 |
| | | 338 M | Asp | OD1 | 2.9 |
| | | 338 M | Asp | OD2 | 3.5 |
| | | 339 M | Ser | OG | 3.1 |
| | | 375 M | Gly | CA | 3.3 |
| | | 339 M | Ser | C | 3.9 |
| N8 | N | 365 M | Gly | CA | 3.8 |
| | | 339 M | Ser | O | 3.3 |
| | | 367 M | Gly | C | 3.8 |
| | | 367 M | Gly | O | 2.8 |
| | | 368 M | Cys | CA | 3.8 |
| | | 338 M | Asp | CG | 3.5 |
| | | 338 M | Asp | OD1 | 3.7 |
| | | 338 M | Asp | OD2 | 2.7 |

The below formula (I-NO) shows the atom numbering used in Table 18 for the compound (S)-2-{2-[5-(5-carbamimidoyl-1H-benzoimidazol-2-yl)-6,2′-dihydroxy-5′-sulfamoyl-biphenyl-3-yl]acetylamino}-succinic acid:

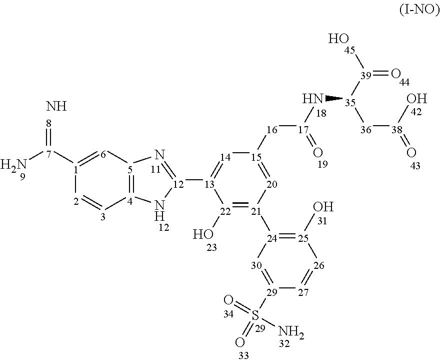

The example demonstrate that (S)-2-{2-[5-(5-carbamimidoyl-1H-benzoimidazol-2-yl)-6,2′-dihydroxy-5′-sulfamoyl-biphenyl-3-yl]acetylamino}-succinic acid stabilizes factor FVIIa by interactions with the FVIIa active site including the catalytic amino acid residues His193 and Ser344 (SEQ ID NO: 1) and the adjacent active site pocket.

The above examples illustrate practice of the invention. These examples are included for illustrative purposes only and are not intended in any way to limit the scope of the invention claimed.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A liquid pharmaceutical composition comprising:
a Factor VIIa polypeptide;
a buffering agent suitable for keeping pH in the range of from about 5.5 to about 8.5; and
an active site stabilizing agent selected from the group consisting of:
(S)-2-{2-[5-(5-carbamimidoyl-1H-benzoimidazol-2-yl)-6,2′-dihydroxy-5′-sulfamoyl-biphenyl-3-yl]acetylamino}-succinic acid (Formula I), or a pharmaceutically acceptable salt thereof;
(R)-2-{2-[5-(5-carbamimidoyl-1H-benzoimidazol-2-yl)-6,2′-dihydroxy-5′-sulfamoyl-biphenyl-3-yl]acetylamino}-succinic acid (Formula II), or a pharmaceutically acceptable salt thereof;
a mixture of (S)-2-{2-[5-(5-carbamimidoyl-1H-benzoimidazol-2-yl)-6,2′-dihydroxy-5′-sulfamoyl-biphenyl-3-yl]acetylamino}-succinic acid or a pharmaceutically acceptable salt thereof;
and (R)-2-{2-[5-(5-carbamimidoyl-1H-benzoimidazol-2-yl)-6,2′-dihydroxy-5′-sulfamoyl-biphenyl-3-yl]acetylamino}-succinic acid or a pharmaceutically acceptable salt thereof.

2. A composition according to claim 1, wherein the active site stabilizing agent is: (S)-2-{2-[5-(5-carbamimidoyl-1H-benzoimidazol-2-yl)-6,2′-dihydroxy-5′-sulfamoyl-biphenyl-3-yl]acetylamino}-succinic acid, or a pharmaceutically acceptable salt thereof;

3. A composition according to claim 1, wherein the active site stabilizing agent is present in an excess of 5.5-100 μM compared to the concentration of Factor VIIa; or the active site stabilizing agent is present in an excess of ≧20 μM compared to the concentration of Factor VIIa.

4. A composition according to claim 1, wherein the molar ratio between the active site stabilizing agent and FVIIa polypeptide ([active site stabilizing agent]:[FVIIa]) is in the range of 1.25-1.75.

5. A composition according to claim 1, wherein the Factor VII polypeptide is present in a concentration of: about 0.3-200 mg/mL.

6. A composition according to claim 1, having a pH value from 6.0-8.5.

7. A composition according to claim 1, wherein the formulation comprises one or more of: an antioxidant, a tonicity modifying agent, and a surfactant.

8. A composition according to claim 1, wherein the Factor VII polypeptide is selected from the group consisting of human Factor VIIa, recombinant human Factor VIIa, and serum-free recombinant human FVIIa

9. A composition according to claim 1, wherein the Factor VII polypeptide is a Factor VII sequence variant or a Factor VII derivative.

10. A method of treating a Factor VII-responsive bleeding disorder in a patent in need of such treatment, comprising administering to the patient a therapeutically effective amount of a liquid pharmaceutical composition according to claim 1 and a pharmaceutically acceptable carrier.

11. A liquid pharmaceutical composition according to claim 1 for treatment of a Factor VII-responsive bleeding disorder.

12. A method for preparing a liquid pharmaceutical composition according to claim 1, comprising providing the Factor VIIa polypeptide in a solution comprising the buffering agent and the 2-{2-[5-(5-carbamimidoyl-1H-benzoimidazol-2-yl)-6,2′-dihydroxy-5′-sulfamoyl-biphenyl-3-yl]acetylamino}-succinic acid or pharmaceutically acceptable salt thereof.

13. A method for stabilizing Factor VIIa in a liquid aqueous composition, comprising providing the Factor VIIa polypeptide in a solution comprising a buffering agent suitable for keeping pH in the range of from about 5.5 to about 8.5 and 2-{2-[5-(5-carbamimidoyl-1H-benzoimidazol-2-yl)-6,2′-dihydroxy-5′-sulfamoyl-biphenyl-3-yl]acetylamino}-succinic acid or a pharmaceutically acceptable salt thereof.

14. An air-tight container containing a liquid, aqueous pharmaceutical composition as defined in claim 1, and optionally an inert gas.


SEQUENCE LISTING (1 sequence)

SEQ ID NO: 1 - protein, 406 aa, artificial sequences
  SOURCE 1..406: /mol_type=“protein” /note=“Wild-type human coagulation Factor VII” /organism=“artificial sequences”
ANAFLXXLRPGSLXRXCKXXQCSFXXARXIFKDAXRTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP